(19) United States
(12) Patent Application Publication
(10) Pub. No.: US 2026/0096029 A1
(43) Pub. Date: Apr. 2, 2026

(54) EMBEDDING OF A CIRCUIT OF ELECTRONIC COMPONENTS IN AN ELONGATED FLEXIBLE DEVICE

(71) Applicant: Magnisity Ltd., Hod-HaSharon (IL)

(72) Inventors: Benjamin GREENBURG, Hod-HaSharon (IL); Ron BARAK, Tel Aviv (IL); Igi BRIGMAN, Petah Tikva (IL); Dror GARDOSH, Rishon LeTsiyon (IL); Eyal KLEIN, Tel-Aviv (IL)

(73) Assignee: Magnisity Ltd., Hod-HaSharon (IL)

(21) Appl. No.: 19/120,250

(22) PCT Filed: Oct. 13, 2023
(86) PCT No.: PCT/IL2023/051072

§ 371 Date: Apr. 11, 2025

Related U.S. Application Data
(60) Provisional application No. 63/415,696, filed on Oct. 13, 2022.
(60) Provisional application No. 63/536,465, filed on Sep. 4, 2023.

(51) Int. Cl. H05K 1/189; A61B 1/00; A61B 1/05; H05K 1/02

(52) U.S. Cl. CPC H05K 1/189; A61B 1/00124; A61B 1/05; H05K 1/028; A61B 2562/166; A61B 2562/182; H05K 2201/09009; H05K 2201/10121; H05K 2201/10151

(57) ABSTRACT

The invention relates to an elongated device and method of producing thereof. The elongated device having an elongated body and a sensor array having a Flexible Printed Circuits (FPC) and a plurality of electronic components positioned on the FPC. The sensor array is helically wound around the elongated body the plurality of electronic components are aligned in relation to a longitudinal axis of the elongated body.

### RELATED APPLICATION/S

This application claims the benefit of priority of U.S. Provisional Patent Application No. 63/415,696 filed on 13 Oct. 2022 and further claims the benefit of priority of U.S. Provisional Patent Application No. 63/536,465 filed on 4 Sep. 2023, the contents of which are incorporated herein by reference in their entirety.

### FIELD AND BACKGROUND OF THE INVENTION

The present invention, in some embodiments thereof, relates to Flexible Printed Circuits and, more particularly, but not exclusively, to Flexible Printed Circuits for elongated devices.

Certain medical devices exist which combine electronics inside the devices. For example, many endoscopes exist which contain an electrical image sensor at the endoscope's tip, which is usually accompanied by one or more Light Emitting Diodes (LED). In this case, for example, the image sensor and LEDs are usually powered by a power source located externally to the endoscope and residing in a host station and connected to the endoscope using electrical conductors such as isolated electrical wires inside an electrical cable, and the sensor images are streamed to a host station using one or more electrical conductors, such as isolated shielded electrical wires.

Other types of devices exist which make use of passive electronics. For example, in a traditional Electromagnetic (EM) based tracking system, an EM coil-based sensor may comprise an ultra-thin enameled copper wire wrapped around a small magnetic core (for example, ferrite) and placed at the tip of a tracked EM catheter. The wire at its two ends may then be extended in a twisted-pair fashion back to a connected host system, as a differential signal. Usually, each EM coil requires 2 differential wires. A standard 3D EM coil-based sensor consists for example of 3perpendicular coils, which amounts to 6 wires. For a standard multi-sensor EM application, the number of wires grows linearly with the number of EM sensors in the device.

There exist other types of devices and tools which combine electronic components and sensors, such as but not limited to: pressure sensors, strain sensors, force sensors, imaging sensors, etc. Such devices and tools, for example in the medical field, may be: endoluminal ultrasound devices (such as REBUS, IVUS); other endoluminal imaging (such as OCT and spectroscopy devices); ablation devices (such as RF probes, Microwave probes, cryoablation devices); electrical clot and foreign-object retrieval; flexible endoluminal surgical tools; histotripsy and other types of therapeutic ultrasound devices, electrical cauterization. Most electrical devices and tools require electrical powering, connectivity, and hosting of electrical components inside the devices.

Additional background art includes U.S. Pat. No. 11,712,309 disclosing an EM shape sensor which consists of a sensor-array made of multiple discrete digital 3D magnetometers assembled on a Flexible Printed Circuit (FPC). The sensor-array may be embedded in a manual or robotic endoscope (or other tubular device) to enable EM shape sensing of that endoscope.

### SUMMARY OF THE INVENTION

Following is a non-exclusive list including some examples of embodiments of the invention. The invention also includes embodiments which include fewer than all the features in an example and embodiments using features from multiple examples, also if not expressly listed below.
Example 1. An elongated device, comprising:
   a. a handle;
   b. an elongated body comprising a proximal end and a distal end; said proximal end connected to said handle;
   c. one or more sensor arrays, each comprising:
      i. one or more Flexible Printed Circuits (FPCs); and
      ii. a plurality of electronic components positioned on said one or more FPCs;
   said one or more sensor arrays being helically wound around said elongated body; and wherein said plurality of electronic components are align in relation to a longitudinal axis of said elongated body.
Example 2. The elongated device according to example 1, wherein said plurality of electronic components comprise one or more of sensors, capacitors, resistors, integrated circuits, electromagnetic sensors, digital sensors, digital magnetometers and optical sensors.
Example 3. The elongated device according to example 1 or example 2, wherein each of said plurality of electronic components is oriented on said one or more FPCs at an orientation angle in relation with a longitudinal axis of said one or more FPCs.
Example 4. The elongated device according to any one of examples 1-3, wherein said orientation angle is configured so when said one or more FPCs are helically wound around said elongated body, said each of said plurality of electronic components is aligned in relation to said longitudinal axis of said elongated body.
Example 5. The elongated device according to any one of examples 1-4, wherein said orientation angle is opposite to a winding angle of said one or more sensor arrays.
Example 6. The elongated device according to any one of examples 1-5, wherein helically winding of said one or more sensor arrays on said elongated body is characterized by a winding angle.
Example 7. The elongated device according to any one of examples 1-6, wherein said winding angle is defined by an angle between a longitudinal axis of said one or more FPCs and said longitudinal axis of said elongated body.
Example 8. The elongated device according to any one of examples 1-7, wherein said winding angle is of 45 degrees.
Example 9. The elongated device according to any one of examples 1-8, wherein said winding angle is from 30 degrees to 60 degrees.
Example 10. The elongated device according to any one of examples 1-9, wherein said one or more FPCs comprise a proximal end and a distal end; and wherein said proximal end and/or said distal end are characterized by being cut diagonally at angle that is a same angle as said winding angle.
Example 11. The elongated device according to any one of examples 1-10, wherein said elongated body is characterized by a stiffness, and wherein said winding angle affects said stiffness.
Example 12. The elongated device according to any one of examples 1-11, wherein said stiffness increases when said winding angle is closer to 90 degrees.
Example 13. The elongated device according to any one of examples 1-12, wherein said stiffness increases when said winding angle is closer to 0 degrees.
Example 14. The elongated device according to any one of examples 1-13, wherein said winding angle is fixed along the whole length of said elongated body.
Example 15. The elongated device according to any one of examples 1-14, wherein said winding angle changes along the length of said elongated body.
Example 16. The elongated device according to any one of examples 1-15, wherein all of said plurality of electronic components are positioned on one side of said one or more FPCs.
Example 17. The elongated device according to any one of examples 1-16, wherein some electronic components of said plurality of electronic components are positioned on one side of said one or more FPCs, while other electronic components of said plurality of electronic components are positioned on another side of said one or more FPCs.
Example 18. The elongated device according to any one of examples 1-17, wherein said one or more sensor arrays are helically wound around said elongated body having said at least part of said plurality of electronic components facing said elongated body.
Example 19. The elongated device according to any one of examples 1-18, wherein said elongated body comprises one or more openings; and wherein said at least part of said plurality of electronic components facing said elongated body are inserted within said one or more openings.
Example 20. The elongated device according to any one of examples 1-19, wherein said at least part of said plurality of electronic components are shielded from external electrical interference when inserted in said one or more openings.
Example 21. The elongated device according to any one of examples 1-20, wherein said one or more FPCs are adhered to said elongated body by at least one adhesive.
Example 22. The elongated device according to any one of examples 1-21, wherein said one or more FPCs widen around a location where each of said plurality of electronic components are positioned on said one or more FPCs.
Example 23. The elongated device according to any one of examples 1-22, wherein said one or more FPCs narrow between locations where each of said plurality of electronic components are positioned on said one or more FPCs.
Example 24. The elongated device according to any one of examples 1-23, further comprising one or more conductive wirings helically wound around said elongated body.
Example 25. The elongated device according to any one of examples 1-24, wherein a location of said one or more conductive wirings helically wound around said elongated body is different as a location where said one or more sensor arrays are helically wound around said elongated body.
Example 26. The elongated device according to any one of examples 1-25, wherein said one or more conductive wirings are one or more insulated conductive wirings.
Example 27. The elongated device according to any one of examples 1-26, wherein one or more electronic components are connected to said one or more conductive wiring.
Example 28. The elongated device according to any one of examples 1-27, wherein said one or more conductive wirings are printed conductive ink.
Example 29. The elongated device according to any one of examples 1-28, wherein electronic components from said plurality of electronic components are positioned on different FPCs from said one or more FPCs.
Example 30. The elongated device according to any one of examples 1-29, further comprising a dedicated FPC for said optical sensors.
Example 31. The elongated device according to any one of examples 1-30, wherein all of said plurality of electronic components are positioned on a same FPC.
Example 32. The elongated device according to any one of examples 1-31, wherein said one or more FPCs comprise small copper weights.
Example 33. The elongated device according to any one of examples 1-32, wherein said one or more FPCs comprise small holes to allow plastic materials to flow through said one or more FPCs in a reflow process.
Example 34. The elongated device according to any one of examples 1-33, wherein at least some of said plurality of electronic components are SMT components; and wherein said SMT components are soldered to plated holes in said one or more FPCs.
Example 35. The elongated device according to any one of examples 1-34, wherein said one or more FPCs comprise solder mask between solder pads.
Example 36. The elongated device according to any one of examples 1-35, further comprising at least one camera.
Example 37. The elongated device according to any one of examples 1-36, wherein camera's power and clock and data signals are hosted on a same FPC with said one or more sensor arrays.
Example 38. The elongated device according to any one of examples 1-37, wherein camera's power and clock and data signals are hosted on a separate dedicated FPC.
Example 39. The elongated device according to any one of examples 1-38, wherein camera clock and digital/analog data signal are shielded from electrical interference.
Example 40. The elongated device according to any one of examples 1-39, further comprising dedicated camera power and ground planes configured for shielding said elongated device from other camera signals.
Example 41. The elongated device according to any one of examples 1-40, wherein said one or more FPCs are multilayer FPCs.
Example 42. The elongated device according to any one of examples 1-41, wherein said one or more FPCs only contain camera traces but do not contain an actual camera component.
Example 43. The elongated device according to any one of examples 1-42, further comprising an electrical connector attached to at least one of said proximal end and a distal end of said one or more FPCs.
Example 44. The elongated device according to any one of examples 1-43, wherein said one or more FPCs comprise a male connector at least one of said proximal end and a distal end of said one or more FPCs.
Example 45. The elongated device according to any one of examples 1-44, further comprising an Inter-Integrated Circuit (I2C) or Improved Inter-Integrated Circuit (I3C) bus.
Example 46. The elongated device according to any one of examples 1-45, wherein one or more FPCs are connected to a component in said handle.
Example 47. The elongated device according to any one of examples 1-46, wherein said one or more FPCs comprise one or more creases in one or multiple connecting areas between soldered components.
Example 48. The elongated device according to any one of examples 1-47, wherein said one or more creases comprise a Kresling-pattern.
Example 49. The elongated device according to any one of examples 1-48, wherein said one or more creases comprise a concertina-type hinge.
Example 50. The elongated device according to any one of examples 1-49, wherein said one or more FPCs are not used and wherein a printed circuit design is printed directly onto said elongated body of said elongated device.
Example 51. The elongated device according to any one of examples 1-50, wherein said printed circuit design includes the conductors only.
Example 52. The elongated device according to any one of examples 1-51, wherein the printed circuit design includes conductors and components.
Example 53. The elongated device according to any one of examples 1-52, wherein said FPC is helically winded while preserving a flexibility of said elongated body.
Example 54. The elongated device according to any one of examples 1-53, further comprising a plurality of shielded cables of small diameter winded around said elongated body.
Example 55. The elongated device according to any one of examples 1-54, wherein said plurality of shielded cables are twisted as a single twisted set of cables.
Example 56. The elongated device according to any one of examples 1-55, wherein said plurality of shielded cables are twisted in pairs.
Example 57. The elongated device according to any one of examples 1-56, wherein said plurality of shielded cables are enameled copper wires of small diameter.
Example 58. The elongated device according to any one of examples 1-57, wherein a first FPC layer comprises assembled components, and a second FPC layer comprises data signals.
Example 59. The elongated device according to any one of examples 1-58, wherein power and ground signals are laid out on said FPC as two planes.
Example 60. The elongated device according to any one of examples 1-59, wherein power and ground signals are laid out on a top layer.
Example 61. The elongated device according to any one of examples 1-60, wherein each of said digital magnetometers use 4 pads: voltage, ground, clock and data.
Example 62. An elongated device, comprising:
   a. a handle;
   b. an elongated body comprising a proximal end and a distal end; said proximal end connected to said handle;
   c. one or more sensor arrays, each comprising:
      i. one or more printed circuits; and p3 ii. a plurality of electronic components positioned on said one or more printed circuits;
   said one or more sensor arrays being helically wound around said elongated body; and wherein said plurality of electronic components are align in relation to a longitudinal axis of said elongated body,
   wherein a printed circuit design is printed directly onto said elongated body of said elongated device.
Example 63. A method of manufacturing an elongated device comprising one or more sensor arrays; said one or more sensor arrays comprising one or more Flexible Printed Circuits (FPCs) and a plurality of electronic components positioned on said one or more FPCs; the method comprising helically winding one or more sensor arrays around said elongated device;
   wherein said method comprises positioning said plurality of electronic components along said one or more FPCs so when said one or more sensor arrays are wound around said elongated device, said plurality of electronic components are aligned in relation to a longitudinal axis of said elongated device.
Example 64. The method according to example 63, wherein at least one of the FPCs has a spiral shaped FPC design, and is windable as a helix onto an elongated device.
Example 65. The method according to example 63 or example 64, wherein all of said plurality of electronic components are aligned in relation to a longitudinal axis of said elongated device.
Example 66. The method according to any one of examples 63-65, wherein said plurality of electronic components comprise one or more of sensors, capacitors, resistors, integrated circuits, electromagnetic sensors, digital sensors, digital magnetometers and optical sensors.
Example 67. The method according to any one of examples 63-66, further comprising orienting each of said plurality of electronic components on said one or more FPCs at an orientation angle in relation with a longitudinal axis of said one or more FPCs.
Example 68. The method according to any one of examples 63-67, wherein said orientation angle is configured so when said one or more FPCs are helically wound around said elongated device, said each of said plurality of electronic components is aligned in relation to said longitudinal axis of said elongated body.
Example 69. The method according to any one of examples 63-68, wherein said orientation angle is opposite to a winding angle of said one or more sensor arrays.
Example 70. The method according to any one of examples 63-69, wherein said helically winding of said one or more sensor arrays on said elongated body is characterized by helically winding at a winding angle.
Example 71. The method according to any one of examples 63-70, wherein said winding angle is defined by an angle between a longitudinal axis of said one or more FPCs and said longitudinal axis of said elongated body.
Example 72. The method according to any one of examples 63-71, wherein said winding angle is of 45 degrees.
Example 73. The method according to any one of examples 63-72, wherein said winding angle is from 30 degrees to 60 degrees.
Example 74. The method according to any one of examples 63-73, wherein said one or more FPCs comprise a proximal end and a distal end; and wherein said proximal end and/or said distal end are characterized by being cut diagonally at angle that is a same angle as said winding angle.
Example 75. The method according to any one of examples 63-74, wherein said elongated body is characterized by a stiffness, and wherein said winding angle affects said stiffness.
Example 76. The method according to any one of examples 63-75, wherein said stiffness increases when said winding angle is closer to 90 degrees.
Example 77. The method according to any one of examples 63-76, wherein said stiffness increases when said winding angle is closer to 0 degrees.
Example 78. The method according to any one of examples 63-77, wherein said winding angle is fixed along the whole length of said elongated body.
Example 79. The method according to any one of examples 63-78, wherein said winding angle changes along the length of said elongated body.
Example 80. The method according to any one of examples 63-79, wherein said positioning said plurality of electronic components comprises positioning all of said plurality of electronic components on one side of said one or more FPCs.
Example 81. The method according to any one of examples 63-80, wherein said positioning said plurality of electronic components comprises positioning some electronic components of said plurality of electronic components on one side of said one or more FPCs, while positioning other electronic components of said plurality of electronic components on another side of said one or more FPCs.
Example 82. The method according to any one of examples 63-81, wherein said helically winding comprises winding said one or more sensor arrays around said elongated device having at least part of said plurality of electronic components facing said elongated device.
Example 83. The method according to any one of examples 63-82, further comprising adding one or more openings along said elongated device; and further comprising inserting said at least part of said plurality of electronic components facing said elongated body within said one or more openings.
Example 84. The method according to any one of examples 63-83, further comprising shielding from external electrical interference said at least part of said plurality of electronic components by inserting said at least part of said plurality of electronic components into said one or more openings.
Example 85. The method according to any one of examples 63-84, further comprising adhering said one or more FPCs to said elongated device by at least one adhesive.
Example 86. The method according to any one of examples 63-85, further comprising widening said one or more FPCs around a location where each of said plurality of electronic components are positioned on said one or more FPCs.
Example 87. The method according to any one of examples 63-86, further comprising narrowing said one or more FPCs between locations where each of said plurality of electronic components are positioned on said one or more FPCs.
Example 88. The method according to any one of examples 63-87, further comprising helically winding one or more conductive wirings around said elongated device.
Example 89. The method according to any one of examples 63-88, wherein said helically winding one or more conductive wirings is at a location different from a location of said helically winding one or more sensor arrays.
Example 90. The method according to any one of examples 63-89, wherein said one or more conductive wirings are one or more insulated conductive wirings.
Example 91. The method according to any one of examples 63-90, wherein one or more electronic components are connected to said one or more conductive wiring.
Example 92. The method according to any one of examples 63-91, wherein said one or more conductive wirings are printed conductive ink.
Example 93. The method according to any one of examples 63-92, wherein said positioning said plurality of electronic components comprises positioning at least part of said plurality of electronic components on different FPCs from said one or more FPCs.
Example 94. The method according to any one of examples 63-93, further comprising providing a dedicated FPC for said optical sensors.
Example 95. The method according to any one of examples 63-94, wherein said positioning said plurality of electronic components comprises positioning all of said plurality of electronic components on a same FPC.
Example 96. The method according to any one of examples 63-95, further comprising adding small copper weights to said one or more FPCs.
Example 97. The method according to any one of examples 63-96, further comprising adding to said one or more FPCs small holes to allow plastic materials to flow through said one or more FPCs in a reflow process.
Example 98. The method according to any one of examples 63-97, wherein at least some of said plurality of electronic components are SMT components; and further comprising soldering said SMT components to plated holes in said one or more FPCs.
Example 99. The method according to any one of examples 63-98, wherein said one or more FPCs comprise solder mask between solder pads.
Example 100. The method according to any one of examples 63-99, further comprising adding at least one camera.
Example 101. The method according to any one of examples 63-100, further comprising hosting camera's power and clock and data signals on a same FPC with said one or more sensor arrays.
Example 102. The method according to any one of examples 63-101, further comprising hosting camera's power and clock and data signals on a separate dedicated FPC.
Example 103. The method according to any one of examples 63-102, further comprising shielding camera clock and digital/analog data signal from electrical interference.
Example 104. The method according to any one of examples 63-103, further comprising providing dedicated camera power and ground planes configured for shielding said elongated device from other camera signals.
Example 105. The method according to any one of examples 63-104, wherein said one or more FPCs are multilayer FPCs.
Example 106. The method according to any one of examples 63-105, wherein said one or more FPCs only contain camera traces but do not contain an actual camera component.
Example 107. The method according to any one of examples 63-106, further comprising an electrical connector attached to at least one of said proximal end and a distal end of said one or more FPCs.
Example 108. The method according to any one of examples 63-107, further comprising providing said one or more FPCs with a male connector on at least one of said proximal end and a distal end of said one or more FPCs.
Example 109. The method according to any one of examples 63-108, further comprising providing an Inter-Integrated Circuit (I2C) or Improved Inter-Integrated Circuit (I3C) bus.
Example 110. The method according to any one of examples 63-109, further comprising connecting said one or more FPCs to a component in a handle.
Example 111. The method according to any one of examples 63-110, further comprising providing said one or more FPCs with one or more creases in one or multiple connecting areas between soldered components.
Example 112. The method according to any one of examples 63-111, wherein said one or more creases comprise a Kresling-pattern.
Example 113. The method according to any one of examples 63-112, wherein said one or more creases comprise a concertina-type hinge.
Example 114. The method according to any one of examples 63-113, wherein said method comprises printing a printed circuit design directly onto said elongated device instead of using said one or more FPCs.
Example 115. The method according to any one of examples 63-114, wherein said printed circuit design includes the conductors only.
Example 116. The method according to any one of examples 63-115, wherein the printed circuit design includes conductors and components.
Example 117. The method according to any one of examples 63-116, wherein said winding is performed while preserving a flexibility of said elongated device.
Example 118. The method according to any one of examples 63-117, wherein said winding is performed manually.
Example 119. The method according to any one of examples 63-118, wherein said winding is performed by a winding machine.
Example 120. The method according to any one of examples 63-119, wherein said winding machine comprises an adhesive dispenser and said method comprises by said adhesive dispenser, before said winding, applying adhesive to said electronic circuit and/or applying adhesive to said elongated device.
Example 121. The method according to any one of examples 63-120, wherein said one or more FPCs are twisted about their own axis before being winded on said elongated device.
Example 122. The method according to any one of examples 63-121, wherein said winding is performed on a template elongated device thereby generating a winded electronic circuit; and said method further comprises transferring said winded electronic circuit into said elongated device.
Example 123. The method according to any one of examples 63-122, wherein said applying adhesive to said electronic circuit is performed after said winding is performed on said template elongated device.
Example 124. The method according to any one of examples 63-123, wherein said plurality of sensors are positioned to said one or more FPCs after said one or more FCPs are helically winded on said elongated device.
Example 125. The method according to any one of examples 63-124, wherein said plurality of sensors are soldered onto said one or more FCPs prior said one or more FCPs are helically winded on said elongated device.
Example 126. The method according to any one of examples 63-125, further comprising reflowing soldering pads after said winding.
Example 127. The method according to any one of examples 63-126, wherein said reflowing is performed by one or more of a soldering iron, a hot air gun and a reflow oven.
Example 128. The method according to any one of examples 63-127, further comprising automatically assembling said plurality of electronic components using Pick-and-Place machines.
Example 129. The method according to any one of examples 63-128, further comprising manually assembling said electronic components.
Example 130. A sensor array according to example 1.
Example 131. A method of manufacturing a sensor array according to the method according to example 63.
Example 132. A winding machine configured to wind an electronic circuit into an elongated device, comprising:
   a. a rotor configured for rotating said elongated device along a longitudinal axis of said elongated device;
   b. a feeder for providing said electronic circuit during a winding action;
   c. a controller, comprising instructions for rotating said rotor and for moving said feeder, both at a certain velocity.
Example 133. The winding machine according to example 132, wherein said winding machine comprises an adhesive dispenser configured to apply adhesive to said electronic circuit before winding said electronic circuit.
Example 134. The winding machine according to example 132 or example 133, wherein said electronic circuit comprises a Flexible Printed Circuit (FPC) and a plurality of sensors attached to said FPC; and wherein said FPC is fed into said winding machine by said feeder.
Example 135. The winding machine according to any one of examples 132-134, wherein said plurality of sensors on said FPC are facing said elongated device when said electronic circuit is helically winded around said elongated device.
Example 136. The winding machine according to any one of examples 132-135, wherein facing said plurality of sensors towards said elongated device shields said plurality of sensors from external electrical interference.
Example 137. The winding machine according to any one of examples 132-136, wherein conductive wiring is fed into the winding machine.
Example 138. The winding machine according to any one of examples 132-137, wherein said conductive wiring is insulated conductive wiring.
Example 139. The winding machine according to any one of examples 132-138, wherein said feeder is fixed and said elongated device is rotated along its axis.
Example 140. The winding machine according to any one of examples 132-139, wherein said elongated device is fixed and said feeder is rotated around said device.
Example 141. The winding machine according to any one of examples 132-140, wherein said feeder is configured for twisting said electronic circuit about its own axis; and wherein said electronic circuit is twisted about its own axis before being winded on said elongated device.
Example 142. The winding machine according to any one of examples 132-141, wherein said feeder feds said electronic circuit at an angle in relation to said elongated device.
Example 143. The winding machine according to any one of examples 132-142, wherein said angle is a fixed angle.
Example 144. The winding machine according to any one of examples 132-143, wherein said fixed angle is a 45 degrees angle.
Example 145. The winding machine according to any one of examples 132-144, wherein said angle is an angle that changes while performing said winding.
Example 146. The winding machine according to any one of examples 132-145, wherein said angle changes between 30 degrees and 60 degrees.
Example 147. The winding machine according to any one of examples 132-146, wherein said controller comprises instructions for synchronizing said winding process such that a linear velocity and an angular velocity are synced according to said winding angle.
Example 148. The winding machine according to any one of examples 132-147, further comprising monitoring means for monitoring said winding.
Example 149. A spiral shaped FPC design, which is winded to a helix onto an elongated device.
Example 150. The spiral shaped FPC design according to example 149, wherein the FPC is designed with a diameter between 50 mm to 100 mm.
Example 151. The spiral shaped FPC design according to example 149 or example 150, wherein spiral FPCs are packed into an FPC panel in a hexagonal tiling fashion.
Example 152. A hexagonal spiral shaped FPC.
Example 153. An FPC used to contribute to the mechanical attributes of a device.
Example 154. The FPC according to example 153, wherein the FPC is used instead of support structure, such as braid or coil, in an elongated device, such as catheter.

According to an aspect of some embodiments of the present invention there is provided an electronic circuit that is applied in helical winding around a center (along a longitudinal axis) of an elongated flexible device. In some embodiments, a potential advantage of using a helical winding is that it potentially preserves flexibility of the elongated flexible device while providing electrical conductivity.

According to some embodiments of the invention, the winding is performed manually.

According to some embodiments of the invention, the winding is performed by a winding machine.

According to some embodiments of the invention, the winding machine includes an adhesive dispenser configured for providing an adhesive to the electronic circuit.

According to some embodiments of the invention, the adhesive is applied to the electronic circuit before winding.

According to some embodiments of the invention, the adhesive is applied to the electronic circuit after winding.

According to some embodiments of the invention, the winding machine includes a feeder device.

According to some embodiments of the invention, Flexible Printed Circuit (FPC) is fed into the winding machine via the feeder device.

According to some embodiments of the invention, a small copper weight per layer is used to increase FPC mechanical flexibility (for example, 0.5 oz copper)

According to some embodiments of the invention, FPC contains small holes to allow the plastic materials to flow through the FPC in a reflow process.

According to some embodiments of the invention, FPC is cut diagonally in the distal end in the same angle as the winding angle.

According to some embodiments of the invention, FPC is fed upside-down, such that when the electronic circuit is wound the FPC shields the components from external electrical interference.

According to some embodiments of the invention, the substrate has cut-outs so when the FPC is wound upside-down components fit into the cut-outs.

According to some embodiments of the invention, conductive wiring is fed into the winding machine.

According to some embodiments of the invention, the conductive wiring is insulated.

According to some embodiments of the invention, the feeder is fixed, and the elongated flexible device is rotated along its axis.

According to some embodiments of the invention, the elongated flexible device is fixed, and the feeder is rotated around the device.

According to some embodiments of the invention, the fed substance is twisted about its own axis before winding.

According to some embodiments of the invention, the angle between the winding device and feeder is controlled.

According to some embodiments of the invention, the angle is fixed, for example 45°, to produce fixed winding pitch.

According to some embodiments of the invention, the angle is varied, to produce a helix of different winding angles (which correspond to different winding pitches).

According to some embodiments of the invention, the winding machine comprises a controller that synchronizes the winding process such that the linear velocity and the angular velocity are synced according to the winding angle.

According to some embodiments of the invention, the winding is supervised visually, for example, by an external camera providing a Top view, to control winding parameters.

According to some embodiments of the invention, the winding is supervised visually, for example, by an external camera providing a Top view, to control winding parameters such that electrical components (such as sensors, capacitors etc.) would be positioned in predetermined locations and angles along the catheter after winding (for example, would align on a single axis along the catheter).

According to some embodiments of the invention, winding is performed on a template elongated device which is not the final assembled device, such as a mandrel, before being transferred to the final device.

According to some embodiments of the invention, glue is applied to components after winding.

According to some embodiments of the invention, electronic components are assembled into the electronic circuit.

According to some embodiments of the invention, electronic components are soldered onto an FPC prior to its winding.

According to some embodiments of the invention, the electronic components are positioned and oriented on the FPC such that after winding they all lie on the same axis.

According to some embodiments of the invention, the electronic components are oriented on the FPC in an angle opposite to the winding angle, such that after being wound they occupy minimal space and relieve the strain on their soldered pads.

According to some embodiments of the invention, the electronic components are oriented on the FPC in an angle that support clockwise winding of the FPC.

According to some embodiments of the invention, the electronic components are oriented on the FPC in an angle that support counterclockwise winding of the FPC.

According to some embodiments of the invention, the FPC widens around components and narrows in the gap between components.

According to some embodiments of the invention, SMT components are soldered onto plated holes in the FPC.

According to some embodiments of the invention, SMT components are soldered onto plated holes in the FPC which are not tented (not covered by solder mask) on the flip side of the FPC, to allow air flow during soldering such that the SMT's solder would flow through the plated holes out to the flip side.

According to some embodiments of the invention, FPC has solder mask between solder pads for component.

Optionally, electronic components are soldered onto an FPC component after its winding.

According to some embodiments of the invention, electrical components are assembled onto a pre-wrapped FPC, such that the soldered pads take the shape of a curved tube onto which they're wound.

According to some embodiments of the invention, the component's soldering pads (for example, ball grid array (BGA) solder bumps) can be further reflowed after being helically wrapped, for example using a soldering iron, a hot air gun, a reflow oven or any other suitable method.

According to some embodiments of the invention, electronic components are soldered onto conductive wiring.

According to some embodiments of the invention, conductive wires can be printed using conductive ink

According to some embodiments of the invention, electronic components are automatically assembled using Pick-and-Place machines.

According to some embodiments of the invention, electronic components are assembled manually.

According to some embodiments of the invention, electronic components are assembled along one or more specific axes.

According to some embodiments of the invention, components are assembled along one axis.

According to some embodiments of the invention, the assembly is supervised visually, for example, by an external camera providing a Top view, to control placement of each component to be placed on a selected axis along the device.

According to some embodiments of the invention, one or more electronic components are assembled onto one or more dedicated separate circuits.

According to some embodiments of the invention, a shielded cable of small diameter is used.

According to some embodiments of the invention, wires are twisted as a single twisted set of wires

According to some embodiments of the invention, wires are twisted in pairs to provide shielding to the carried electrical signals.

According to some embodiments of the invention, wires are enameled copper wires of small diameter (for example, wires of thickness 36AWG or thinner).

According to some embodiments of the invention, multiple FPCs are used.

According to some embodiments of the invention, a component is a camera.

According to some embodiments of the invention, the components combine digital and analog image sensor, on the same circuit.

According to some embodiments of the invention, the camera cable is wrapped helically inside the device.

According to some embodiments of the invention, a camera is connected through traces on the same FPC as the sensor-array, or on a separate dedicated FPC.

According to some embodiments of the invention, the camera power (e.g., VCC and GND) and clock and data signals are hosted on a same FPC with the sensor-array, or on a separate dedicated FPC.

According to some embodiments of the invention, camera clock and digital/analog data signal are shielded to protect them from electrical interference.

According to some embodiments of the invention, dedicated camera power and ground planes are used for shielding of other camera signals (clock and data).

According to some embodiments of the invention, the camera and sensors share the same power and ground planes to reduce FPC size.

According to some embodiments of the invention, the FPC is a multilayer FPC, for example, 4-layers FPC, such that the addition of camera signals does not increase it in width.

According to some embodiments of the invention, the sensor-array and camera traces are separated into two sub-FPCs so that the sensor-array and camera traces each lies on a dedicated FPC.

According to some embodiments of the invention, the final FPC only contains the camera traces (for example, power, ground, clock and data) but does not contain the actual camera component.

According to some embodiments of the invention, the FPC includes both camera traces and camera component pads, and the camera is then assembled directly on the FPC.

According to some embodiments of the invention, the wound circuit is connected using an electrical connector to a PCB component.

According to some embodiments of the invention, the wound circuit is connected to a PCB component using a male connector at one end of the circuit.

According to some embodiments of the invention, an I2C (Inter-Integrated Circuit) or I3C (Improved Inter-Integrated Circuit) bus is used.

According to some embodiments of the invention, the circuit comprises more than one layer. One layer (for example, top layer) contains assembled components, and a second layer (for example, bottom layer) contains the data signals (for example, clock and data in case of an I2C bus).

According to some embodiments of the invention, the power and ground signals are laid out on the circuit as two planes, for example on the top layer, to reduce resistance of power signals as well as to shield the data signals on the other layer.

According to some embodiments of the invention, a digital magnetometer uses 4 pads: voltage, ground, clock and data.

According to some embodiments of the invention, a digital magnetometer is a BGA component which consists of 4 BGA bumps as solder pads.

According to some embodiments of the invention, the circuit is longer than the elongated device.

According to an aspect of some embodiments of the present invention there is provided an FPC that is manufactured with one or a plurality of creases in one or multiple connecting areas between soldered components.

According to some embodiments of the invention, Kresling-pattern is used.

According to some embodiments of the invention, a concertina-type hinge, such as found in the bending section of drinking straws, is used.

According to an aspect of some embodiments of the present invention there is provided a printed circuit design that is printed directly onto a flexible material of an elongated flexible device, such that basic flexibility of the device is preserved.

According to some embodiments of the invention, the printed design includes the conductors only.

According to some embodiments of the invention, the printed design includes conductors and components.

According to an aspect of some embodiments of the present invention there is provided a spiral shaped FPC design, which is wound to a helix onto an elongated device.

According to some embodiments of the invention, the spiral shaped FPC is designed with a diameter between 50 mm to 100 mm

According to some embodiments of the invention, spiral FPCs are packed into an FPC panel in a hexagonal tiling fashion,

According to an aspect of some embodiments of the present invention there is provided a hexagonal spiral shaped FPC.

According to an aspect of some embodiments of the present invention there is provided an FPC used to contribute to the mechanical attributes of a device.

According to some embodiments of the invention, FPC is used instead of support structure, such as braid or coil, in an elongated device, such as catheter.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic representation of an exemplary endoluminal device, according to some embodiments of the invention;

FIG. 2 is a schematic representation of an exemplary sensor array, according to some embodiments of the invention;

FIGS. 3a-3b are schematic representations of exemplary general incorporation of sensor arrays into elongated devices, according to some embodiments of the invention;

FIG. 4a is a schematic representation of an elongated device having a sensor array in a wound or helix geometrical configuration, according to some embodiments of the invention;

FIG. 4b is a schematic representation of an exemplary FPC wrapped into a cylindrical shape, according to some embodiments of the invention;

FIG. 4c is a schematic representation of an exemplary FPC having wider areas, according to some embodiments of the invention;

FIG. 5 is a schematic representation of an exemplary spiral FPC, according to some embodiments of the invention;

FIG. 6 is a schematic representation of an exemplary hexagonal tiling configuration of a plurality of spiral FPCs in an FPC panel, according to some embodiments of the invention;

FIG. 7 is a schematic representation of an exemplary FPC having a diagonal distal end, according to some embodiments of the invention;

FIG. 8 is a schematic representation of an exemplary pre-wound helical FPC;

FIGS. 9a-9b are schematic representations of through-hole mounting method and surface mounting method, respectively.

FIGS. 10a-10b are schematic cross-sectional representations of exemplary components of exemplary FPC, according to some embodiments of the invention;

FIG. 11 is a schematic front view cross-section of an exemplary configuration of an exemplary elongated device, according to some embodiments of the invention;

FIG. 12 is a schematic representation of an exemplary method of incorporation of a sensor array into an elongated body of an elongated device with the sensors facing down, according to some embodiments of the invention;

FIG. 13 is a schematic representation of an exemplary automated winding machine, according to some embodiments of the invention; and

FIG. 14 is a schematic representation of an exemplary elongated device comprising a sensor array and a camera, according to some embodiments of the invention.

### DESCRIPTION OF SPECIFIC EMBODIMENTS OF THE INVENTION

The present invention, in some embodiments thereof, relates to Flexible Printed Circuits and, more particularly, but not exclusively, to Flexible Printed Circuits for elongated devices.

### Overview

An aspect of some embodiments of the invention relates to embedding of electrical components, as well as a Flexible Printed Circuit (FPC), in an elongated flexible device. In some embodiments, a sensor array, which comprises discrete sensing elements, is assembled as an FPC or is assembled directly on the elongated device itself. In some embodiments, the FPC and/or conducting wires are wrapped helically inside a device's wall. In some embodiments, the device is an endoscope and the device's wall defines an endoscope's working channel. In some embodiments, the FPC and/or conducting wires are wrapped helically around the endoscope's working channel. In some embodiments, the assembly is then reflowed inside the device's wall or covered with polymer tube or polymer heat shrink tube. In some embodiments, components assembled along the length of the device are positioned such that they all lie on the same axis inside the device, or such that they lie linearly in groups. In some embodiments, assembled components can be further reflowed or glued or fixed by heat shrink tubing after being wrapped inside the device. In some embodiments, a potential advantage of reflowed or glued or fixed by heat shrink tubing is to potentially relieve strain on their soldering pads. In some embodiments, in the case of an endoscope, for example, the conductors or FPC may be longer than the length of the endoscope (for example, 1 meter longer) such that it extends from the endoscope's (or in general, the device's) proximal end to the endoscope's handle. In some embodiments, a potential advantage of helically winding the electronic circuit around a center of an elongated flexible device is to potentially preserve flexibility of the elongated device while providing electrical conductivity. In some embodiments, the FPC may be manufactured in many configurations, such as a straight long FPC, or as a spiral FPC, which is unpacked and wrapped in an assembly process. In some embodiments, the FPC optionally contains shielded conductors, for example, for a digital or analog endoscopic camera. In some embodiments, the camera's signals and EM sensing elements' signals may co-exist on the same FPC. In some embodiments, the FPC's distal end may further contain a camera. In some embodiments, the camera may be spatiality manipulated, such as through folding, and molded into the endoscope's tip as part of the assembly process. In some embodiments, the final assembly can optionally contain both camera and sensing elements (which can be SMT components) and are automatically assembled using, for example, Pick-and-Place machines. As used herein, the terms SMT (Surface Mount Technology) and SMD (surface-mount devices) are used as interchangeable terms, and they mean “the entire technology of mounting and soldering electronic components onto a FPC or PCB”. In some embodiments, optionally, the FPC or conductors are automatically wrapped inside an endoscope using robotic assembly machines.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

An aspect of some embodiments of the invention relates to providing an elongated flexible device and a method for embedding of electrical components, as well as a Flexible Printed Circuit (FPC) in an elongated flexible device.

### Exemplary Endoluminal Device

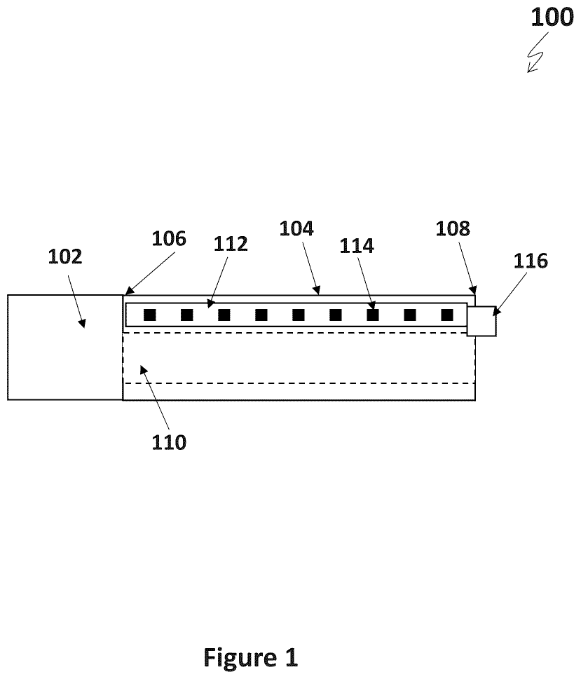

Referring now to FIG. 1, showing a schematic representation of an exemplary endoluminal device, according to some embodiments of the invention. In some embodiments, an exemplary endoluminal device 100 comprises:
1. A handle 102 at a proximal end of the endoluminal device 100;
2. An elongated body 104, comprising a proximal end 106 and a distal end 108. The proximal end 106 connected to the handle 102. In some embodiments, optionally, the elongated body 102 comprises a working channel 110 (area between dashed lines);
3. A sensor array 112 comprising a plurality of electrical components 114 (referred hereinafter just as “sensors 114”), for example sensors, capacitors, resistors, etc. It should be understood that over the following paragraphs, the term “sensors 114” refers to any electrical component 114 needed to be used on the sensor array 112. In some embodiments, the sensors 114 are spread along the elongated body 104; and
4. Optionally, an additional sensor 116, for example an optical sensor and/or camera, located at a most distal end of the elongated body 102.

In FIG. 1, the sensor array 112 is schematically shown as a straight “strip” with the plurality of sensors 114. It should be understood that the sensor array 112 is not limited to a straight configuration, as will be further explained below. Additionally, the components 114 are shown as squares, but it should be understood that the components can have any geometrical form: triangle, square, rectangle, etc.

In some embodiments, the sensors 114 are Electromagnetic sensors (EM sensors), and optionally, the sensors 114 are digital 3D magnetometers. In some embodiments, the sensor array 112 is configured for sensing position and/or shape of the elongated body 102.

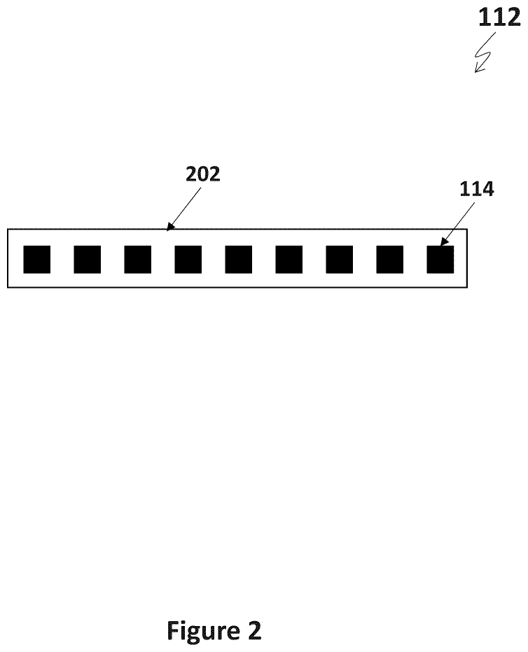

Referring now to FIG. 2, showing a schematic representation of an exemplary sensor array 112, according to some embodiments of the invention. In some embodiments, as mentioned above, an exemplary sensor array comprises a plurality of sensors 114. In some embodiments, the plurality of sensors are mounted on a continuous elongated Flexible Printed Circuit (FPC) 202. In some embodiments, components 114 are assembled on both sides of the FPC. In some embodiments, components 114 and/or the additional sensor 116 can be connected to any side of the FPC.

### Exemplary Embodiments of General Incorporation of Sensor Arrays Into Elongated Devices

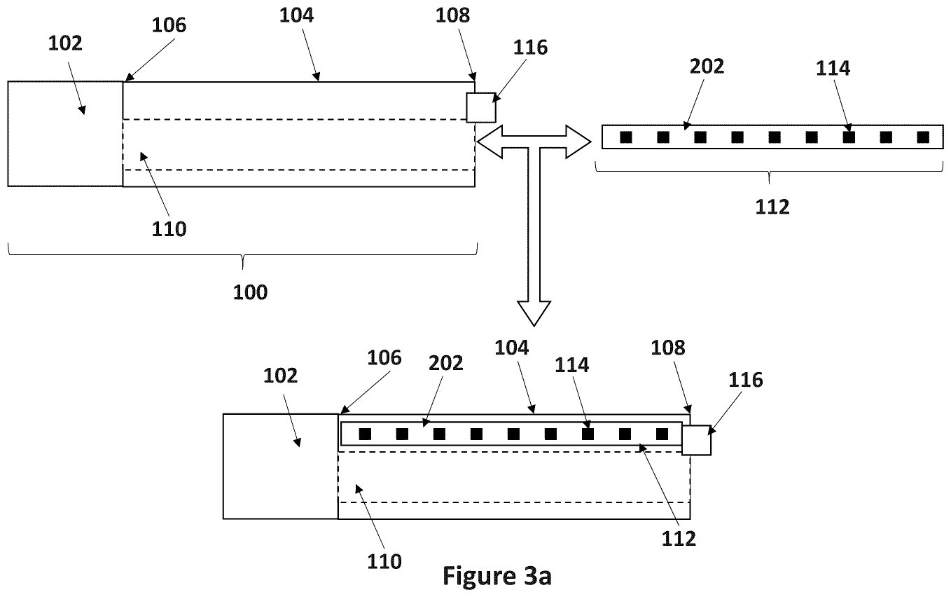

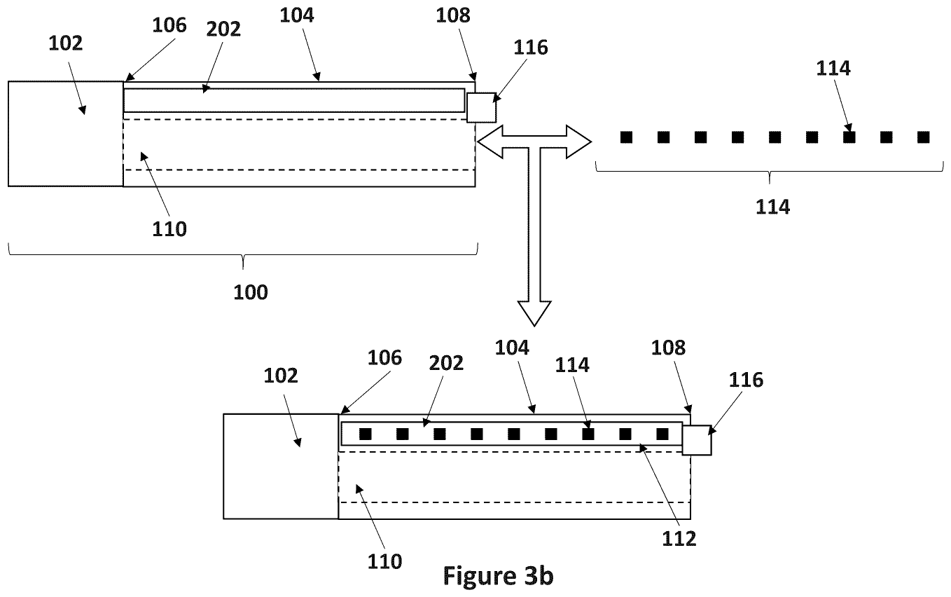

Referring now to FIGS. 3a-b, showing schematic representations of exemplary general incorporation of sensor arrays into elongated devices, according to some embodiments of the invention.

In some embodiments, a sensor array 112, comprising an FPC 202 with a plurality of sensors 114, is prepared and/or manufactured separately from the elongated device 100. In some embodiments, the sensor array 112 is then mounted onto the elongated device 100, as schematically shown for example in FIG. 3a.

In some embodiments, an elongated device 100 is manufactured with an incorporated FPC 202, but without a plurality of sensors 114. In some embodiments, the plurality of sensors 114 are then incorporated into the FPC 202 of the elongated device, as schematically shown for example in FIG. 3b. In some embodiments, some of the components 114 are assembled on the FPC 202 prior to its incorporation into the device 100 while other components 114 may be assembled after incorporation of the FPC 202 into the device 100.

In some embodiments, as mentioned, the FPC 202 and/or conducting wires are wrapped (optionally, helically—see below) inside a device's wall, optionally around an endoscope's working channel. In the following paragraphs, the general term “device” or “elongated device” will be used. It should be understood that the terms refer to any elongated device having a wall, for example an endoscope having a wall, the wall defining a working channel of the endoscope.

In some embodiments, the assembly (FPC 202 and sensors 114) is then encapsulated inside the device's wall, for example by polymer reflow process, covering with polymer tube or with polymer heat shrink tube. Alternatively, by heat shrink or by using a dipping process. In some embodiments, the encapsulation provides a biocompatible layer preventing non-biocompatible components from coming in contact with tissues during use. In some embodiments, the encapsulation layer provides electrical insulation to the encapsulated FPC 202 and components 114. In some embodiments, the rectangular cross section of the FPC 202 allows it to bend in one direction while making it stiffer in the perpendicular directions. In some embodiments, the encapsulation provides EM shielding to the encapsulated FPC 202 and components 114.

In some embodiments, in either method of incorporation, assembled components can be optionally further reflowed or glued or fixed by heat shrink tubing after being wrapped inside the device or incorporated into the FPC 202. In some embodiments, a potential advantage of performing further reflowed or glued or fixed by heat shrink tubing is to potentially relieve strain on the component's soldering pads. In some embodiments, the outer layer comprises a composite design, for example, including a braid or other reinforcement.

### Exemplary Embodiments of Geometrical Incorporation of Sensor Arrays Into Elongated Devices

In some embodiments, a sensor array 112, whether being manufactured separately and then incorporated or sensors being incorporated into an elongated device, is positioned on an elongated device with a certain geometrical configuration.

In some embodiments, an exemplary sensor array 112 is geometrically positioned in a straight configuration, as schematically shown in FIG. 1. In some embodiments, having the sensor array 112 in a straight geometrical configuration provides the elongated device with a relative stiff line along one side of the elongated device, as the FPC with the sensors are all arranged along a straight line on one side of the elongated device. While the inventors have found that this straight geometrical configuration could be useful for some applications, it might be somehow limiting for other applications, as will be further explained below.

In a standard EM tracked device, the number of wires in the EM tracked device grows linearly with the number of used sensors when using a standard EM coil-based sensor. For that, and other reasons, in most EM tracked devices, a single EM position and orientation sensor is used (usually made of 3 perpendicular EM coils). Using multiple coil-based EM sensors in a single device requires the handling of many wires (for example, 3 twisted-pairs per EM sensor), which can be cumbersome or impractical under certain footprint constraints.

To solve this, and for other reasons, U.S. Pat. No. 11,712,309 discloses an EM shape sensor, comprising a sensor array, made of a plurality of discrete sensor elements. Each sensor element may be an SMT 3D digital magnetometer, assembled on a Flexible Printed Circuit (FPC). While resolving the problem of finite capacity to thread a growing number of wires through limited space, it may nonetheless pose a potential problem of maintaining the mechanical flexibility and desired footprint of a device. It has been found by the inventors that to embed the FPC inside an endoscope, special care needs to be taken to account for electrical and mechanical constraints. For example, if the FPC were to be embedded inside the endoscope's wall as an elongated straight FPC, the resulting device's mechanical flexibility and steerability may be impaired due to the FPC's inability to stretch axially, and even more so—laterally, to allow endoscope bending. Alternatively, if the FPC is placed in an open lumen so it is free to move axially, the device may maintain its flexibility however higher footprint may be required, and the sensors are not fixed to a single point of the device and therefore their function might be compromised, for example the accuracy of shape-sensing may be reduced. Therefore, in some embodiments, the sensor array 112 is geometrically positioned is a different manner on the elongated device.

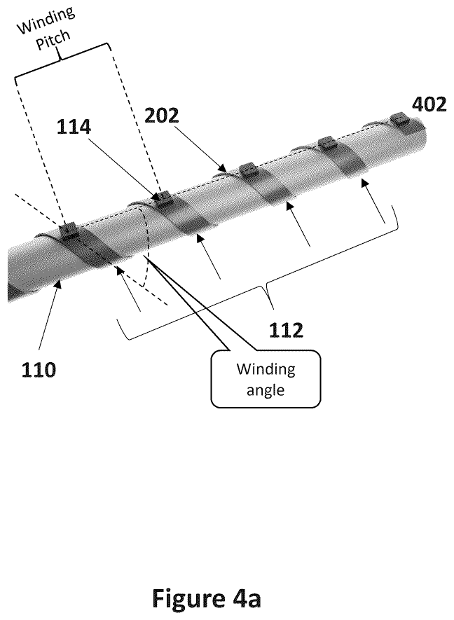

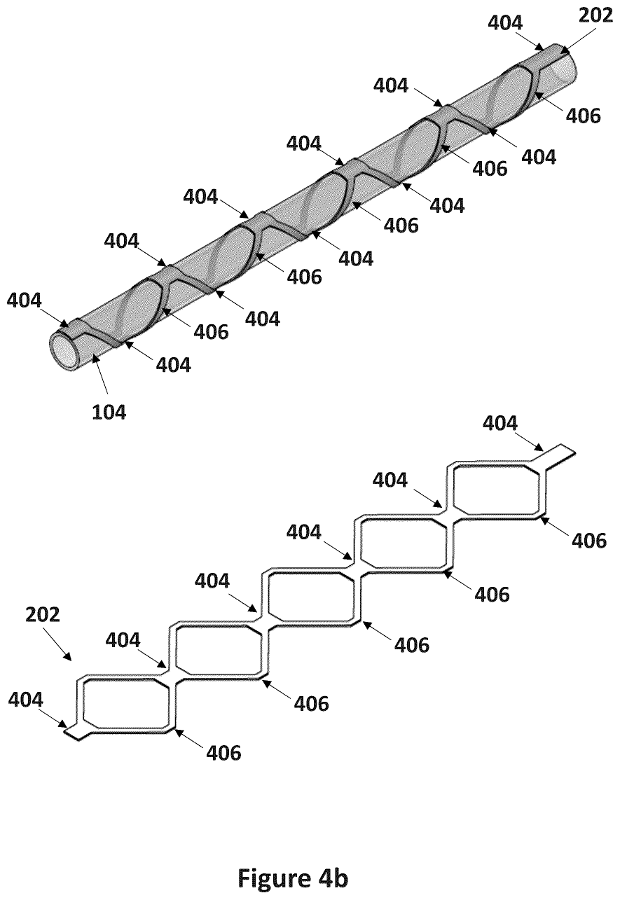

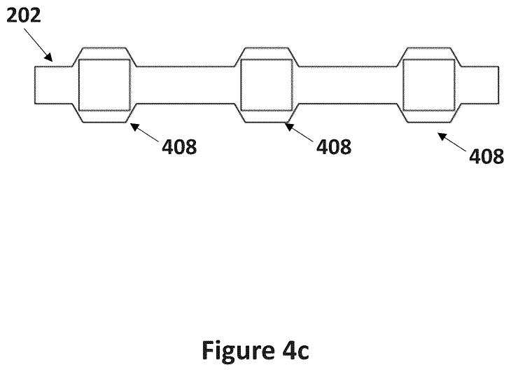

Referring now to FIG. 4a, showing a schematic representation of an elongated device having a sensor array in a wound or helix geometrical configuration, according to some embodiments of the invention.

In some embodiments, an exemplary sensor array 112 is geometrically positioned in a wound or helix configuration, as schematically shown in FIG. 4a. FIG. 4a shows an elongated device 100 and a sensor array 112, which comprises the FPC 202 with a plurality of sensors 114.

Referring now to FIG. 4b, showing a schematic representation of an elongated device having an FPC wrapped in a cylindrical geometrical configuration, according to some embodiments of the invention.

In some embodiments, an exemplary FPC 202 is geometrically wrapped or bent over an elongated device 100 into a cylindrical shape, as schematically shown in FIG. 4b. FIG. 4b shows an elongated device 104 and an FPC 202.

In some embodiments, exemplary FPCs 202 are generated having specialized forms and/or geometries. In some embodiments, a rhombus-like pattern comprises central wide areas 404 configured for receiving electrical components 114 (not shown), and connected between them by one or more connecting bridges 406.

In some embodiments, a potential advantage of using the wrapping and winding methods disclosed herein is that it potentially overcomes the abovementioned problems. In some embodiments, the FPC is able to bend in all directions, surmounting the FPC's inability to stretch. In some embodiments, an FPC containing EM sensor array can sustain twisting about its own axis to create flexibility of the twisted FPC in all axes. In some embodiments, the rectangular cross section of the FPC allows the FPC to bend in one direction while making it stiffer in the perpendicular direction. In some embodiments, the FPC is embedded in an endoscope's wall or in a closed end catheter, such that the EM shape tracked catheter is steerable in all directions.

In some embodiments, a potential advantage of using the wrapping or winding methods disclosed herein is that it potentially allows for the control and minimization of the Cost of Goods Sold (COGS). For example, in some embodiments, using the wrapping or winding methods potentially contributive to a reduction in manual labor in the manufacturing and assembly of a tracked medical device, and thus to a significantly reduced COGS.

In some embodiments, as mentioned above, a potential advantage of using the wrapped or wound up FPC 202 is that it allows maneuvering the elongated device 100 to all directions since the FPC 202 itself can withstand the bending required when the elongated device is maneuvered. In some embodiments, additionally or alternatively, a plurality of creases are added to the FPC 202 in connecting areas between soldered components. In some embodiments, a potential advantage of adding creases is that it potentially preserves the bending capabilities of the elongated device. In some embodiments, there can be one or more creases, and the creases can be in one or more of: a single axis, alternating axis, in 3D crease patterns, for example Kresling-pattern and/or for example as concertina-type hinge, such as found in the bending section of drinking straws. Referring now to FIG. 4c, showing a schematic representation of an exemplary FPC 202 having wider areas, according to some embodiments of the invention. In some embodiments, the FPC 202 widens 408 at dedicated locations, for example, at locations where electronic components 114 are positioned, to support the assembly of these components, and/or to provide enough space for FPC traces to bypass those components, and/or to improve mechanical support of the assembled components or for any other suitable reason.

In the following paragraphs, principles and methods for providing an elongated device having a sensor array with a wound or helix configuration.

### Exemplary Principles of an Exemplary FPC

In some embodiments, as mentioned above, an exemplary sensor array 112 comprises an FPC 202 with a plurality of sensors 114. In the following paragraphs a specific example will be used to allow a person having skills in the art to understand the invention. The example is not intended to be limiting in any way. In some embodiments, the sensor array 112 is an EM shape sensor consisting of a plurality of discrete sensor elements, each of which may be for example a 3D digital magnetometer, assembled on a single FPC 202. In some embodiments, in the case of digital magnetometers, all or some of them share a same digital bus inside the FPC 202. In some embodiments, a potential advantage of having all or some of the sensors on a same bus is that it potentially reduces the number of signals required on the FPC 202 to communicate with the plurality of sensor elements, for example, to as few as 1 signal. In some embodiments, an I2C (Inter-Integrated Circuit) or I3C (Improved Inter-Integrated Circuit) bus is used, which may require as few as 2 signals per bus (clock and data). In some embodiments, an additional 2 wires may be used to power the sensors (for example, voltage and ground). In some embodiments, the FPC 202 comprises of two layers. In some embodiments, one layer (for example, top layer) contains the assembled sensors and the second layer (for example, bottom layer) contains the data signals (for example, clock and data in case of an I2C bus). In some embodiments, each digital magnetometer may have 4 pads: voltage, ground, clock and data. In some embodiments, the power and ground signals may be laid out on the FPC 202 as two planes, for example on the top layer, to reduce resistance of power signals as well as to shield the data signals on the other layer.

### Exemplary Technical Characteristics of an Exemplary FPC

In some embodiments, the FPC 202 comprises a length of for example >20 cm long, or >50 cm long, or >1 m long; and comprises a width of for example <2 mm or <1.5 mm or <1mm. In some embodiments, the FPC 202 comprises a thickness of for example <0.13 mm or <0.1 mm. In some embodiments, the FPC 202 uses small copper weight per layer to increase its mechanical flexibility, for example, 0.5 oz copper. In some embodiments, optionally, the FPC 202 contains small holes, orifices or protrusions to allow the plastic materials to flow through the FPC 202 during a reflow process. In some embodiments, additionally or alternatively, it allows adhesive to flow during reflow process.

In some embodiments, as mentioned above, in order to maintain steerability of the final assembled device, the FPC 202 is wrapped helically inside the wall of the elongated device (e.g. endoscope), around the endoscope's working channel, as schematically shown for example in FIG. 4.

### Exemplary Angles of Winding of an Exemplary Sensor Array in a Wound or Helix Geometrical Configuration

In some embodiments, winding a sensor array 112 along the elongated body 104 of an elongated device 100 is characterized by a winding angle, which is defined as an angle between a longitudinal axis of the sensor array 112 and a longitudinal axis of the elongated body 104. In some embodiments, the sensor array 112 is wound to have fixed winding angles, thereby having a uniform winding pitch. In some embodiments, the sensor array 112 is wound to have different winding angles, thereby having a varying winding pitch along the elongated device. In some embodiments, the larger the winding angle θ is between the FPC 202 and the axis of the elongated body 104 of the elongated device 100 (for example, closer to 60 degrees, or closer to 70 degrees, or closer to 90 degrees), the smaller is the winding pitch and the elongated device is characterized by having an increased flexibility. In some embodiments, in the case of smaller winding pitch, the FPC needs to be longer to support the wrapping of the FPC 202 around the entire or most of the length of the elongated device (for example by a factor of 1/cos θ). In some embodiments, additionally, a smaller pitch amounts in increased stiffness due to the increased amount of FPC material in the device (copper, polyimide, components etc.). In some embodiments, on the other hand, the smaller the winding angle θ is between the FPC 202 and the axis of the elongated body 104 of the elongated device 100 (closer to 0 degrees), the larger is the winding pitch and the elongated device is stiffer (at least in one bending axis). This happened for example, when the sensor array 112 is positioned almost only along one side of the elongated device (meaning having a winding angle close to 0 degrees), therefore providing a “stiffening component” to one side only of the elongated device. In some embodiments, an intermediate winding angle, for example a winding angle of about 45 degrees, provides a good compromise between the above considerations: it provides the necessary amount of flexibility and/or stiffness to the device while not increasing the FPC length too much (it requires an FPC longer by a factor of \sqrt{2}). In some embodiments, a winding angle in the range of from about 30 degrees to about 60 degrees is used. For example, a winding angle of about 60 degrees is used at the tip of the elongated device, where flexibility is usually mostly required. In some embodiments, the winding angle is then gradually decreased to about 30 degrees as the FPC 202 is wrapped along the elongated body 104 of the elongated device 100 towards the proximal end of the elongated device, where flexibility is usually less required. In some embodiments, a potential advantage of using a dynamic winding pitch along the elongated device is that it potentially allows the FPC 202 not to be too long (for example, <1.5 meters long) while maintaining flexibility at the tip of the elongated device. In some embodiments, the FPC 202 is covered with insulation after the assembly of the components 114. In some embodiments, the insulation is applied selectively over exposed electrical contacts. In some embodiments, the insulation consists of a conformal coating such as acrylic, PU, parylene, epoxy. In some embodiments, insulation consists of insulated tape.

### Exemplary Use of the FPC to Affect Mechanical Features of the Elongated Device

In some embodiments, as mentioned above, the FPC 202 is embedded inside the device, for example inside an endoscope, in order to modify one or more of the pushability, the torquability, the steerability, kink resistance and other mechanical properties of that endoscope. In some embodiments, in this case, other mechanical properties of the elongated device can be modified to account for the mechanical properties of the embedded FPC 202. For example, if a braid is used in the construction of the elongated device, a thinner braid can be used, for example, with less pushability. Then, the addition of the embedded FPC will compensate for the “missing” pushability while reducing the final footprint of the elongated device. For example, the device's outer-diameter will not increase or will only increase slightly due to the embedding of the FPC in the device. In some embodiments, the FPC 202 is used not just to add electrical features to a device (such as a sensor-array or a camera) but also to deliberately affect the mechanical properties of the device. In some embodiments, the FPC 202 acts as reinforcement to the device's wall, potentially replacing the use of braid or other reinforcement altogether while providing the desired mechanical properties of the device.

### Exemplary Principles for the Manufacture of Exemplary Sensor Arrays

In some embodiments, a process of manufacturing an exemplary sensor array 112 comprises manufacturing a dedicated FPC 202.

In some embodiments, the FPC 202 may be manufactured in many configurations, such as a straight long FPC 202, or as a spiral FPC 202 which is unpacked and wrapped in an assembly process.

In some embodiments, optionally, the FPC 202 contains shielded conductors, for example, for a digital or analog endoscopic camera. In some embodiments, the signals of the camera and the signals of the plurality of sensor 114 are transmitted along the same FPC 202. In some embodiments, optionally, during the manufacturing process of the FPC, a camera is connected to a distal end of the FPC 202. In some embodiments, the camera may be spatiality manipulated, such as through folding, and may be molded into the elongated device's tip as part of the assembly process. In some embodiments, the final assembly can optionally contain both camera and sensors (see above “Exemplary embodiments of general incorporation of sensor arrays into elongated devices”). In some embodiments, the components (camera and/or sensors) are surface mount device (SMD) components, and are optionally automatically assembled, for example, using Pick-and-Place machines.

In some embodiments, the FPC or conductors can be automatically wrapped inside an elongated body of an elongated device using automated assembly machines.

In some embodiments, the FPC 202 is manufactured in medium length, for example, <30 cm long. Standard FPC manufacturing processes, as well as Pick-and-Place machines, commonly support FPC of length <50 cm, so that the manufacturing and assembly of a long FPC (for example, of length >50 cm) may be expensive and increase COGS. However, several medical devices are of length >50 cm, specifically, several endoscopes require a length >50 cm, for example, several manual and robotic bronchoscopes. In this case, an FPC of size <50 cm cannot be wrapped around the full length of such devices. In addition, using for example a winding angle θ=45°, the FPC length should increase by a factor of \sqrt{2}, as described above, such that for example, to cover a device of length 71 cm, the FPC needs to be at least 1 meter long.

In some embodiments, the FPC 202 can be of medium length, which is supported by standard FPC manufacturing and assembly processes, for example, about 30 cm long. In this case, the FPC 202 can be wrapped around the distal part of a device. In some embodiments, since the FPC 202 may not be long enough to cover the entire length of the elongated device 100, electrical isolated wires are soldered to pads which are exposed on the proximal side of the FPC 202, and the wires can be extended to the handle 102 (where they can be connected to controller electronics etc.). In some embodiments, optionally, the wires continue to wrap around the elongated device in a helical manner to retain the device's flexibility, similarly to the helix of the FPC 202. In some embodiments, optionally, the wires are twisted as a single twisted set of wires, or are twisted in pairs to provide some level of shielding to the carried electrical signals. In some embodiments, optionally, the wires are enameled copper wires of small diameter (for example, wires smaller than 36AWG wires).

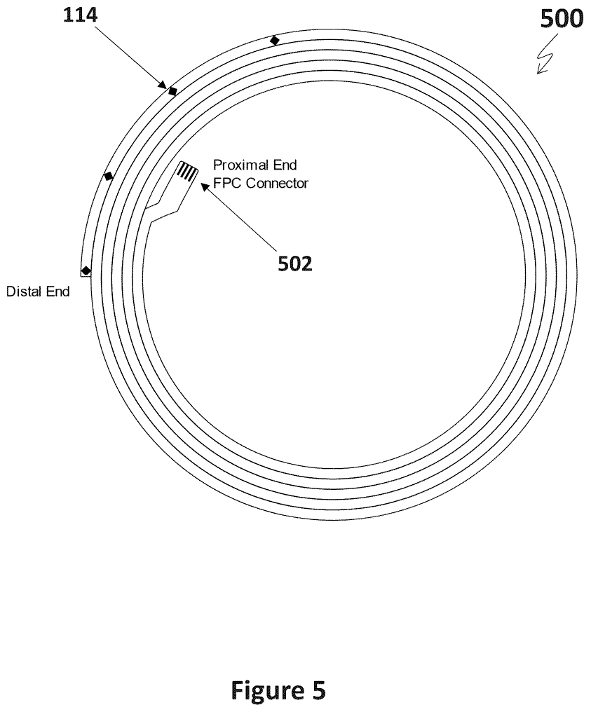

Since soldered wires involve manual labor which increases COGS, and since simple soldered wires have uncontrolled shielding and other electrical characteristics such as resistance, capacitance and inductance (even when twisted together), it is preferable to use a single FPC 202 along the entire length of the device. In some embodiments, to produce a FPC 202 long enough to be wrapped around a long device in 45 degrees, or in a varying 30-60 degrees (for example, FPC of length 1 meter to be wrapped around 70 cm long device, or FPC of length 1.3 meters to be wrapped around 80 cm long device), in some embodiments, a spiral shaped FPC can be used, as schematically shown in FIG. 5.

Referring now to FIG. 5, showing a schematic representation of an exemplary spiral FPC, according to some embodiments of the invention.

In some embodiments, the FPC 202 comprises a spiral configuration (spiral FPC denoted with the reference number 500). For example, in an exemplary spiral configuration, the FPC 500 comprises five spiral turns. In some embodiments, any suitable number of turns is applicable under various embodiments and according to the needs. In some embodiments, sensors 114 are placed on the FPC 500 so as to match a certain rotation angle of the FPC 500, for example to reside on the same axis after winding of the FPC 500 on a shaft, for example, a working channel. In some embodiments, an exemplary FPC 500 (or any FPC 202 disclosed herein) optionally comprises a connector 502 located at the proximal end of the FPC 500. In some embodiments, the spiral FPC 500 is manufactured as a spiral which fits inside an FPC panel of standard size. For example, the spiral can be of diameter 70 mm, or of diameter less than 80 mm, and five spiral turns can be performed, so that the FPC 500, when extracted, will be of 1 meter length, or 1.3 meters length, or 1.5 meters length or any other suitable length. In some embodiments, the spiral can also be for example of diameter 100 mm and 3¼-spiral turns can be made to obtain the full length of one meter. In some embodiments, the larger the diameter of the spiral, the easier it is to wrap it around a device while maintaining a fixed winding angle. In some embodiments, an exemplary diameter of the spiral is from about 50 mm to about 100 mm, optionally from about 30 mm to about 200 mm, optionally from about 10 mm to about 500 mm. In some embodiments, a potential advantage of using a diameter of from about 50 mm to about 100 mm is that it potentially provides a good compromise between reducing the spiral's curvature for winding to manufacturing a rather densely packed FPC panel, since when using a spiral of large diameter, a large percentage of the FPC manufactured panel is potentially left unused.

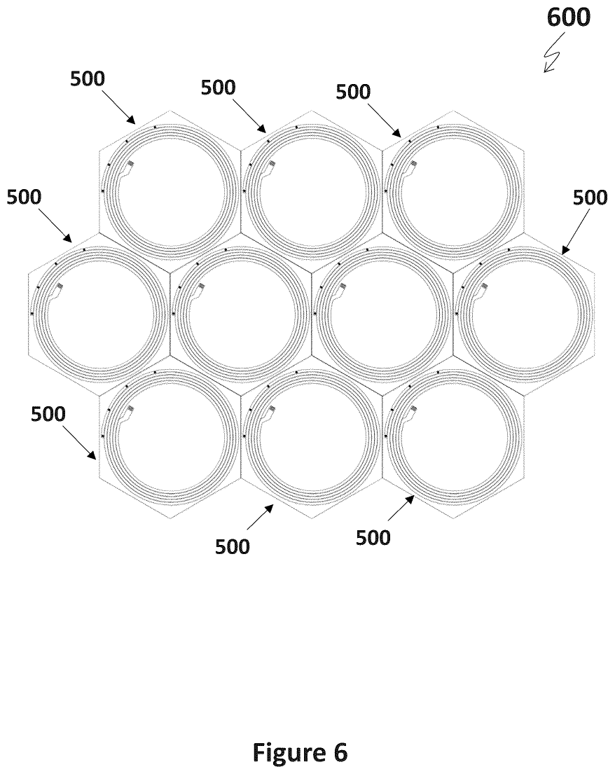

Referring now to FIG. 6, showing a schematic representation of an exemplary hexagonal tiling configuration of a plurality of spiral FPCs in an FPC panel, according to some embodiments of the invention. In some embodiments, an exemplary FPC panel 600 is divided in hexagonal tiles, each containing a spiral FPC 500. In some embodiments, a potential advantage of using a hexagonal tiling is that most of the panel is used and there is less waste of prime panel material. In some embodiments, other geometrical configurations are used, for example, rectangular, triangular, pentagonal, etc. In some embodiments, instead of designing the FPC as a round spiral, the FPC is designed directly as a hexagonal spiral.

In some embodiments, as mentioned above, at the proximal end of the FPC there is a connector 502. In some embodiments, a potential advantage of adding a connector 502 at the proximal end of the FPC is that it potentially further reduces manual labor in the embedding process of the FPC inside an elongated device. In some embodiments, the connector 502 is connected without soldering to a FPC compatible connector residing on electronics rigid PCB board in the elongated device's handle 102, or an elongated device's mounting interface.

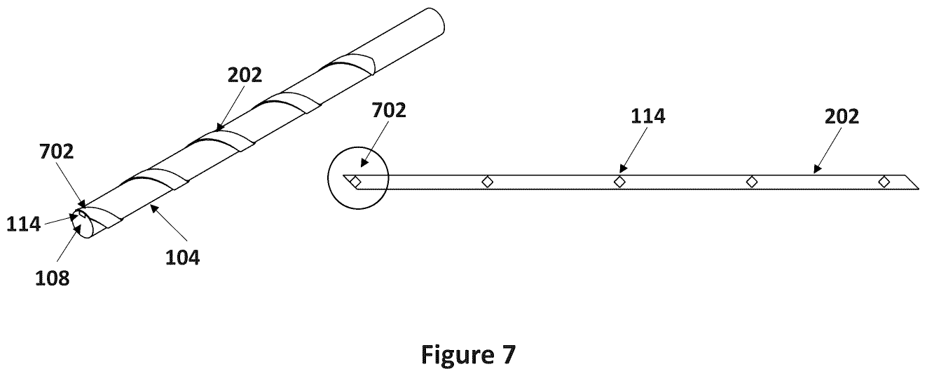

Referring now to FIG. 7, showing a schematic representation of an exemplary FPC having a diagonal distal end, according to some embodiments of the invention.

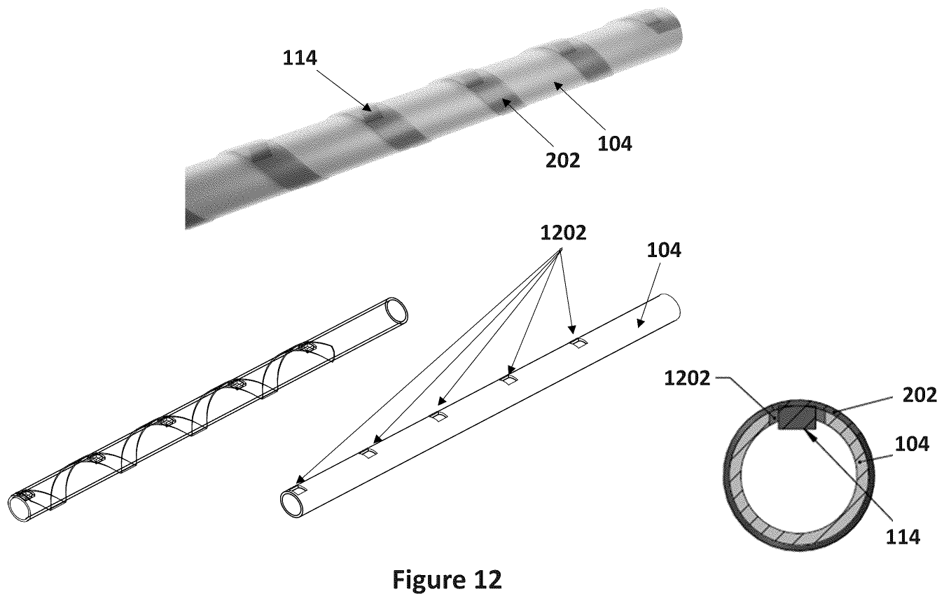

In some embodiments, the FPC 202 may include features to direct and assist in the winding process. In some embodiments, for example, the distal end of the FPC may end in a diagonal 702 with an angle a corresponding to the winding angle, so when this edge is aligned with the distal end 108 of the shaft 104 the axis of the FPC is naturally aligned with the correct winding angle. In FIG. 7, the sensors 114 on the FPC 202 are facing the shaft 104 when the FPC 202 is mounted on the shaft 104 (see also below in relation to FIG. 12).

In some embodiments, optionally, the soldering pads of the components are further reflowed after being helically wrapped, for example using a soldering iron, a hot air gun, a reflow oven or any other suitable method. In some embodiments, a potential advantage of performing a reflowing is that it potentially relieves strain on the soldered pads of the electrical components assembled on the FPC (including the sensor elements). In some embodiments, performing a reflowing potentially allows the soldered pads to take the shape of the curved tube on which they're wrapped, thus relieving the strain on the pads.

In some embodiments, heating applied to the wrapped FPC can be high heat to reflow the solder material, or low heat that to only relieve residual stresses in the solder material caused by the winding process. In some embodiments, specific temperatures change according to component and solder material used, for example, high temperature range required for reflow would be above the melting temperature of the solder material and low range would be below it. For example, for a lead-free solder material commonly used in the biomedical field, which has a melt point of 217° C., a high range for reflow could be between 250-260° C., and a lower range for stress relief could be 190-200° C.

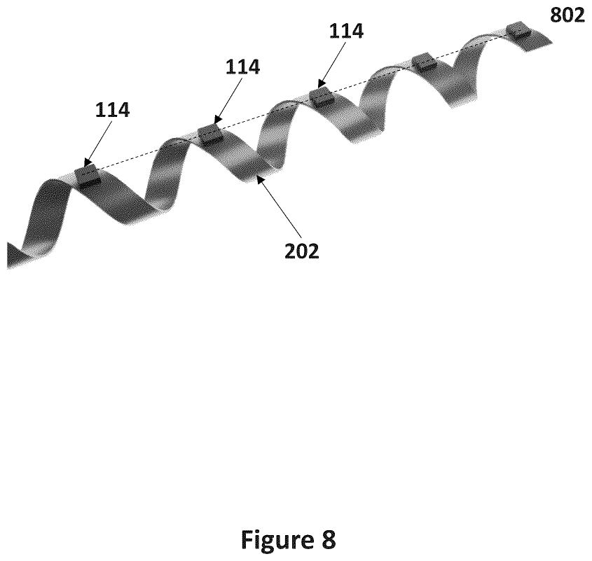

In some embodiments, alternatively, the FPC can be wrapped around some other tube, such as a mandrel, which is used for the assembly of the FPC. In some embodiments, after being wrapped, the electrical components (for example sensors 114) are directly assembled on the wrapped FPC, such that the soldered pads take the shape of a curved tube to which they're wrapped. In some embodiments, such mandrel comprises a similar diameter as the device itself onto which the wound FPC will be mounted. In other embodiments, the mandrel comprises a larger or smaller diameter as the device itself onto which the wound FPC will be mounted. In some embodiments, after being assembled with components, the assembled FPC can be extracted from the mandrel, in its helix form (“pre-wrapped” form), as schematically shown, for example, in FIG. 8. In some embodiments, the components are assembled to the FPC before being wrapped around the mandrel, and then reheated as described above to release residual strains before being assembled onto the endoscope. In some embodiments, the assembled helical FPC can then be assembled, for example, around an endoscope's working channel (elongated body 104 of elongated device 100), before being encapsulated inside an endoscope's wall. In some embodiments, as mentioned above, the components 114 are aligned on a same longitudinal axis, depicted by dashed line 802.

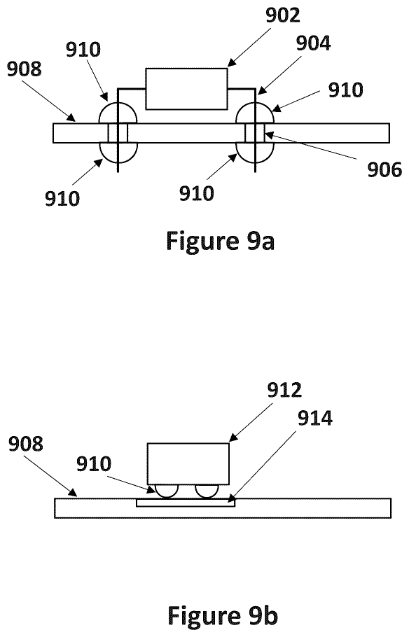

Without being bound to theory, there are two main methods for mounting components onto a PCB: through-hole mounting and surface mounting. Referring now to FIG. 9a, showing a schematic representation of through-hole mounting method for mounting components onto a PCB. In through-hole mounting, dedicated through-hole components 902 comprise a plurality of leads (“legs”) 904 configured to enter holes 906 in the PCB 908. Then, soldering material 910 is used to fix the through-hole components into the PCB. Referring now to FIG. 9b, showing a schematic representation of surface mounting method for mounting components onto a PCB. In surface mounting, dedicated surface-mount components (SMT/SMD) 912 are soldered onto a pad 914 surface of the PCB 908, usually using soldering material 910 located on the bottom part of the surface-mount component 912.

In some embodiments, the FPC 202 utilizes a combination of the two methods.

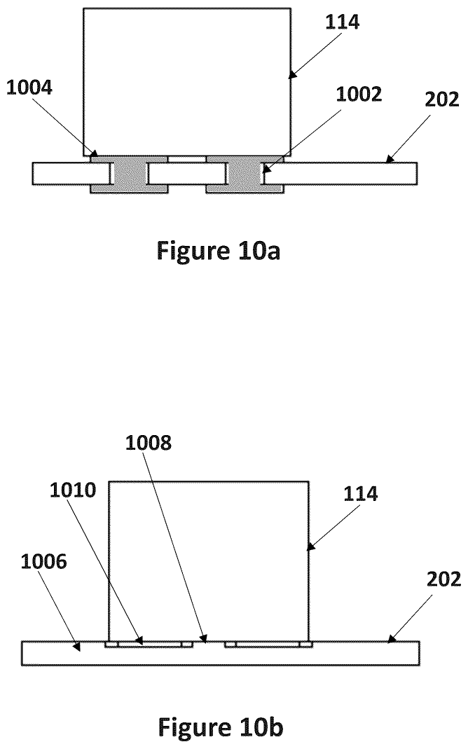

Referring now to FIGS. 10a and 10b, showing schematic cross-sectional representations of exemplary components mounted on exemplary FPC, according to some embodiments of the invention.

In some embodiments, the FPC 202 includes plated through-holes 1002 (also referred to Vias 1002) placed on the pads of the sensors to allow solder material 1004, either from the ball-grid array (BGA) bumps of the component itself or otherwise added, to flow through the through-holes 1002 of the FPC 202, mechanically locking the component 114 to the FPC 202 and potentially improving its durability to withstand the winding process and bending. In some embodiments, the FPC includes local reinforcements in specific locations along the device. In some embodiments, this reinforcement is achieved by adding layers to the FPC, such as layers of polyimide stiffener, or other polymer, metallic or ceramic material. In some embodiments, reinforcements are added after manufacturing of the FPC in a separate process. In some embodiments, reinforcements are locally placed under, near or around components, potentially improving their durability to withstand the winding process and bending. In some embodiments, the reinforcements are positioned in selected locations, potentially assisting in bonding of the FPC onto the device.

In some embodiments, soldering material of the BGA bumps flows into the vias allowing the sensor to lay tight against the FPC, thereby reducing the overall height of the FPC and sensor. In some embodiments, a light pressure may be applied on the components during the reflow process to assist solder material flow into the vias. In some embodiments, excess solder material is removed from the side of the FPC opposite the components after reflow for example by using a soldering iron, a solder removing tool, or by mechanical means. In some embodiments, soldering material of the BGA bumps flowing into the vias provides strong contact between the BGA bumps and the FPC, thus reducing the risk of broken contacts especially during winding of the FPC, where forces are being applied to the pads.

In some embodiments, the solder mask 1006 (or soldermask) is not completely removed from the FPC 202 in the gaps 1008 between solder pads 1010 of the electrical components 114, thereby potentially reducing the risk of solder material flowing between pads and causing short-circuits. In some embodiments, the solder mask is partially or completely removed in the side of the FPC opposite to the components to allow for trapped air to flow outside of the vias while the soldering material of the BGA bumps will be able to flow into the vias. In some embodiments, the solder mask is not completely removed in the side of the FPC opposite to the components. In some embodiments, the solder mask is completely removed on the side of the component in the area directly under the component, potentially allowing it to sink further up to the height of the conductor layer of the FPC.

### Exemplary Arrangement of Components Along the Length of Device

In some embodiments, components, for example the sensors 114, assembled along the length of the device, are positioned on the FPC 202 such that they all lie on the same axis, as schematically shown for example in FIG. 4, depicted by dashed line 402. In some embodiments, optionally, a discrete number of sensors 114 lie linearly in groups.

In some embodiments, optionally, as schematically shown in FIG. 5, the sensors 114 and other sensing elements/components are designed and assembled on the FPC with a rotation angle relative to the FPC axis, which corresponds to the winding angle of the FPC inside and/or on the elongated device. In some embodiments, for example, for a helix winding angle of θ=45°, the discrete components can be designed and assembled with −θ=−45° rotation on the FPC relative to the FPC axis (which depends on the winding direction: clockwise vs. counterclockwise). In some embodiments, the angle of positioning of the components in relation to the longitudinal axis of the FPC changes along the length of the FPC. In some embodiments, a potential advantage of changing the positioning angle of the components on the FPC is that it can be potentially used to match the changes in winding angle of the FPC along the length of the device. In some embodiments, optionally, providing angles to the components relative to the longitudinal axis of the FPC is done regardless of any present spiral or non-spiral configuration of the FPC. In some embodiments, a potential advantage of assembling the components with a rotation angle opposite to that of the winding angle is that it potentially ensures that the components are straight after winding (aligned with the device's axis), as schematically shown for example in FIG. 4. In some embodiments, a potential advantage of keeping the components aligned relative to the device is that it can potentially reduce the forces exerted on the components' soldered pads due to the tight winding of the FPC around the device, thus making the soldered sensor elements and other electrical components more resilient to bending of the device, for example, to withstand 20 mm bending radius, 15 mm bending radius, 10 mm bending radius or any other desired bending radius. In some embodiments, positioning components rotated compared to the axis of the FPC may require increasing the width of the FPC, which may negatively affect the mechanical properties of the device, for example, by increasing its stiffness. Therefore, in some embodiments of the present disclosure, the width of the FPC may change along its length, increasing near and around electrical components to support the assembly of rotated components and decreasing in gaps between components.

In some embodiments, the electrical components 114 positioned on the FPC 202 are positioned so as to occupy as less space as possible on the FPC 202. In some embodiments, a potential advantage of positioning the electrical components 114 so as for them to occupy as less space as possible on the FPC 202 is to potentially reduce the final footprint of the final elongated device, more specifically, the outer-diameter (OD) of the elongated device. In some embodiments, the sensor elements 114 (and other electrical elements such as SMT capacitors, resistors, integrated circuits, etc.) are placed on the FPC 202 such that after being wrapped in a helix they lie on the same axis, as schematically shown for example, in FIGS. 4, 7 and 8. In some embodiments, for a given winding angle θ, and a given winding radius R (the radius around which the FPC is to be wrapped), distances between the sensor elements 114 on the FPC 202 are computed. In some embodiments, the distances between the sensor elements changes along the FPC to match the changing winding angle or radius of winding along the device's length. In some embodiments, a potential advantage of computing the distances between the electrical components is that it potentially guarantees that each sensor element is placed on the same axis after winding the sensor array on the device. For example, sensor elements 114 (and other components) are placed on the FPC 202 in increments of 2πR/ sin θ, where R is the winding radius and θ is the winding angle, as described above. In some embodiments, as mentioned herein elsewhere, the electrical components 114 are oriented on the FPC 202 in an angle opposite to the winding angle, such that, after being wrapped around the endoscope's working channel (elongated body 104 of the elongated device 100), the electrical components 114 will occupy as much less space as possible, and as well as to relieve the strain on their soldered pads. In some embodiments, as mentioned above, electrical components 114 may be non-square components, for example rectangular. In some embodiments, the non-square components are rotated at an angle so, after winding, they will have the minimal cross-section area. For example, a rectangular shaped component may be placed so that after winding its longer side is parallel to the longitudinal axis of the elongated device and its shorter side is parallel to the cross-section plane of the elongated device.

### Exemplary General Arrangement of a Sensor Array on an Elongated Device

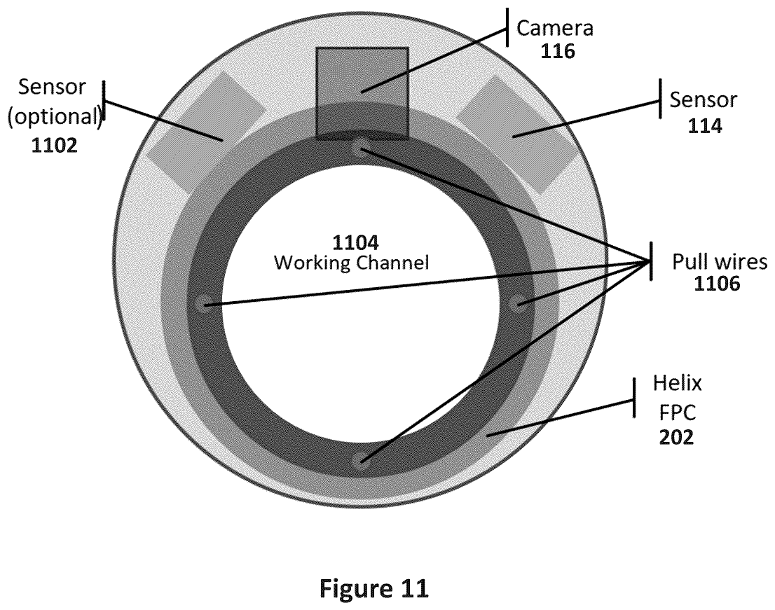

Referring now to FIG. 11, showing a schematic front view cross-section of an exemplary configuration of an exemplary elongated device, according to some embodiments of the invention.

In some embodiments, the sensor elements 114 are positioned and oriented on the FPC 202, such that after being helically wrapped around the elongated body 104 that defines, for example, a working channel of an endoscope, the sensor elements 114 all lie on the same axis, for example, on a single axis out of the working channel 1104 and, optionally, aside to an optional camera 116 or an optional additional sensor 1102. In some embodiments, the longitudinal axis passing through the center of the elongated body 104 (for example the longitudinal axis passing through the center of the working channel) and the longitudinal axis of the overall resulting elongated device (comprising both the elongated body 104, the sensor array 112 and the external cover for the whole elongated device 100) are not coincident (are positioned with an offset between them). In some embodiments, a potential advantage of not coinciding the longitudinal axes is that it potentially leaves more space for the assembled components as well as for the optional camera 116/additional sensor 1102. In FIG. 11, it is also shown four embedded pull wires 1106 alongside with the assembled FPC, which can be advantageous for example for a robotically manipulated endoscope. In some embodiments, an electrical component, for example a sensor or a camera, are positioned in alignment to one of the two or more pull wires 1106. In some embodiments, a potential advantage of aligning between a sensor/camera and one of the two or more pull wires 1106 is that the input received from the sensor/camera is aligned with the bending directions of the elongated device. In some embodiments, electrical components are positioned, along the elongated body, between the two or more pull wires 1106. In some embodiments, a potential advantage of positioning electrical components between the two or more pull wires 1106 along the elongated device is that it potentially reduces the overall cross-sectional footprint of the elongated device. In some embodiments, the camera and sensors are all aligned in one line over one of the pull wires. In some embodiments, the camera and sensors are all aligned in the space between two pull wires. In some embodiments, the camera and sensors can be positioned in any other configuration along the elongated device and in relation to the two or more pull wires 1106.

It should be understood that while FIG. 11 shows a 4 pull-wire design, aligning components as disclosed above, can be done with 2 or more pull wires.

In some embodiments, the camera 116 and/or sensors 114 are located between two adjacent pull wires 1106.

Referring now to FIG. 12, showing a schematic representation of an exemplary method of incorporation of a sensor array into an elongated body of an elongated device, according to some embodiments of the invention.

In some embodiments, as mentioned above, the sensor array 112 is wrapped having the electronic components (for example, sensors 114 or other components) facing the elongated body 104 of the elongated device 100 (“face-down” configuration). In some embodiments, potential advantages of positioning the sensor array this way are one or more of potentially shielding the sensors, potentially reducing the outer-diameter of the complete device (mechanical considerations) and potentially increasing resilience to bending of the soldered components.

In some embodiments, the FPC 202 is wound around a flexible or semi-rigid mechanism located along the elongated body 104 of the elongated device 100, for example, around the deflectable section of an endoscope or around a steerable catheter. In some embodiments, such flexible section may be constructed from separate links, and in other cases from a semi-rigid mechanism, for example laser cut metallic hypotube, similarly designed cylindrical polymer component which includes thin points that can elastically deform to allow this tubular section to bend. In some embodiments, in either of these cases, these bendable mechanisms are designed to include openings 1202 and/or cut-outs (not shown) in the wall of the elongated body 104. In some embodiments, optionally, the openings comprise a size that fits the size of the tridimensional form of the electronic components destined to be inserted therein. In some embodiments, when the FPC 202 is wrapped upside-down as described herein, the components 114 align with the openings 1202 in the flexible mechanism (or the part of the elongated body where the openings 1202 were added) thus not increasing the cross-section footprint of the device, optionally by not more than the FPC thickness (for example, 0.1 mm). In some embodiments, some electrical components are positioned on one side of the FPC 202, while other are positioned on the other side of the FPC 202. In some embodiments, a plurality of electrical components 114 are mounted on a FPC 202 facing up and wound to a dimeter smaller than that of the elongated body 104. In some embodiments, then, the FPC 202 is inserted within the elongated body 104 and then allowed to expand so allow the plurality of components 114 to enter the openings 1202 on the elongated body 104. In this case, the FPC 202 is positioned within the elongated body 104 and not “on” the elongated body.

### Exemplary Use of Conducting Wires Without Substrate

In some embodiments, the bending capabilities are kept by printing the circuit design directly onto the flexible material of the elongated device itself, such that basic flexibility of the device is preserved, for example by printing the circuit using conductive ink.

In some embodiments, a similar helix winding method is used, in which instead of FPC the conducting wires (without the substrate) are directly adhered (by methods such as using glue, resin, heat, cold, US, or other methods of binding) onto the elongated device surface itself. In some embodiments, a potential advantage of doing this is that it potentially allows reduction of mechanical constraints on the elongated device when compared to using FPC. In some embodiments, another potential advantage is that it may be potentially simpler to manufacture in some settings, and potentially allows changes in winding pitch of the conductors. In this scenario, electronic components, sensors and other SMTs are soldered later manually or automatically by machine, in a linear pattern or in any other desired pattern. In some embodiments, optionally, a protective coating may be applied as required. In some embodiments, a potential advantage of this method is that the elongated device itself performs as the FPC substrate for the components and conductors.

### Exemplary Additional Features

In some embodiments, optionally, the conductors or FPC are longer than the length of the elongated device 100, for example, from about 1 meter to about 2 meters longer (in case of a wound FPC and/or wound conductors, the length of the FPC or conductors means the absolute length along one axis, for example the absolute length of a wound FPC along the longitudinal axis of device 100), such that the conductors or FPC extends from the proximal end of the elongated body 104 to handle 102 or to the elongated device's mounting interface (in case for example of a robotic elongated device).

In some embodiments, as mentioned above, the FPC-based sensor array extends from an elongated device's tip up to the handle. In some embodiments, the FPC contains sensing elements (sensors), which can be 3D digital magnetometers. In some embodiments, the FPC may also combine a digital or analog image sensor on the same FPC. In another embodiment, the FPC can be wrapped helically inside the elongated device and the components can be assembled at carefully chosen positions and orientations on the FPC to reduce footprint of the embedded sensor array inside the elongated device. In some embodiments, the FPC can be used for all the electronics inside the elongated device (full EM sensor array for EM shape sensing, digital/analog camera as well as other types of sensors). In some embodiments, the FPC is long enough (for example, 1-2 meter long) such that there is no need for extra wires inside the elongated device. In some embodiments, the FPC is automatically assembled using Pick-and-Place machines.

### Exemplary Thermal Requirements

In some embodiments, electrical components have thermal requirements. For example, the solder material requires high temperatures and specific thermal cycles during a soldering reflow process, while some components have limitations on the maximum temperatures they can withstand or the maximum time they can withstand certain temperatures, before damage of degradation occurs. In some embodiments, other processes, for example thermoplastic polymer reflow commonly used in steerable shaft manufacturing, have other thermal requirements, for example minimum temperatures and duration to allow proper flow of the polymer. In some embodiments, the polymer reflowed material and solder material are selected so that the reflow temperature of the polymer material is lower than the melting/soldering temperature of the solder material, which is in turn lower than the allowed temperature of the electrical components. In some embodiments, the electrical components are protected with a high melting-point material such as high temperature epoxy, to protect them and the soldering material during the polymer reflow process.

While throughout the disclosure focus is given to the winding of FPC around medical devices to enable EM shape sensing in the field of endoscopes and catheters, it should be appreciated that similar application may enable any sensing in any other elongated flexible device. For example, an FPC containing sensor-array can be embedded in a helical manner inside an elongated device for general use, for example, in a VR/AR tracked wire for training or simulations, or for example in a robotic arm and its control mechanisms. Additional examples of sensors that may be similarly integrated into a device using such methods are for example imaging sensors, thermometers, flow meters such as velocimeters and others, ultrasonic transducers and receivers, radiation emitters and radiation detectors, pressure and strain sensors, piezoelectric or other force sensors, and other types of sensors.

As used herein with reference to quantity or value, the term “about” means “within ±20 % of”.

The terms “comprises”, “comprising”, “includes”, “including”, “has”, “having” and their conjugates mean “including but not limited to”.

The term “consisting of”means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc. ; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find support in the following examples.

### EXAMPLES

Reference is now made to the following examples, which together with the above descriptions illustrate some embodiments of the invention in a non limiting fashion.

In some embodiments, an assembled FPC is helically wrapped around a device (for example, around an endoscope's working channel) manually. In some embodiments, the FPC is wrapped with a winding angle of about θ=45° or with a varying winding angle of, for example, 30-60 degrees, such that each assembled component (specifically, each sensor element) is positioned along the same axis after winding, as schematically shown for example in FIG. 4. In some embodiments, keeping a fixed winding angle while positioning each sensor on the same device's axis provides a level of control to the winding process. In some embodiments, it allows an assembly worker to supervise the winding process, using each sensor element as a reference or an anchor for the winding. Alternatively, in another embodiment, to reduce COGS, the winding of the FPC can be done automatically by a winding machine.

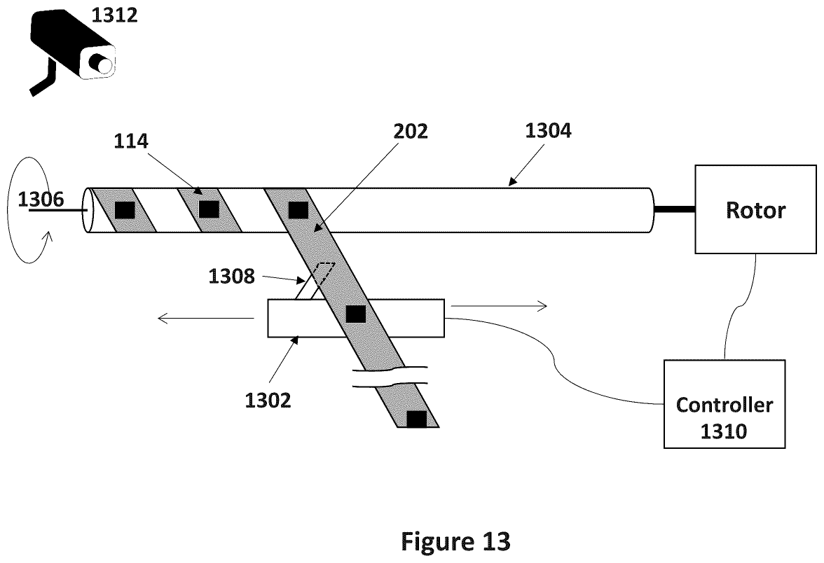

Referring now to FIG. 13, showing a schematic representation of an exemplary automated winding machine, according to some embodiments of the invention. In some embodiments, one or more FPCs 202 can be fed into the feeder slider 1302 for example in one or more reels (not shown), containing the FPCs 202. In some embodiments, the device 1304 around which the FPC 202 is to be wrapped can be rotated around its axis 1306 at a controlled angular velocity synchronized with the feeder 1302 linear movement velocity. In some embodiments, the automatic winding machine can include adhesive dispenser 1308 to apply adhesive to the FPC 202 or the device 1304. In some embodiments, the FPC 202 is held at a fixed angle, for example, at 45° or a variable controlled angle, for example, 30-60 degrees, to allow variable pitch winding. In some embodiments, the automatic winding machine may also have the device 1304 fixed to the automatic winding machine and have the feeder 1302 rotate (and move linearly) around (and along) the device axis 1306. In some embodiments, the winding process is controlled by a controller 1310 which synchronizes the linear velocity with the angular velocity according to the winding angle. In some embodiments, optionally, the process is supervised visually, for example, by an external camera 1312 providing a top view, to make sure that each sensor element is placed on the same axis along the device. In some embodiments, this can be done using automatic image processing techniques. In some embodiments, the automatic winding machine can use the detected sensor positions in the image as feedback to the winding process, for example, to slightly rotate or translate the reel containing the FPC to slightly increase or decrease the winding pitch.

In some embodiments, a similar winding machine is used to wind one or more pairs of conductive wires, instead of FPC 202, maintaining the same abilities to change winding angle, resulting in any fixed or changing winding pitch. In some embodiments, after winding the wires, components are then soldered manually or automatically, so they lie on the same axis or on different axes.

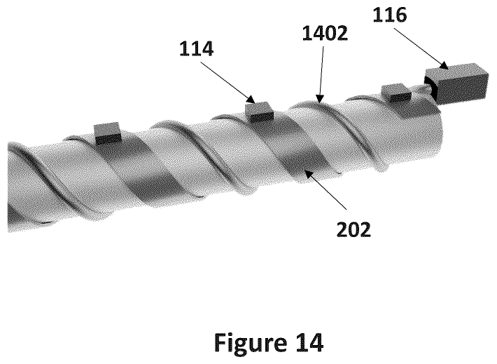

Referring now to FIG. 14, showing a schematic representation of an exemplary elongated device comprising a sensor array and a camera, according to some embodiments of the invention. In some embodiments, as mentioned above, the final elongated device contains a camera 116. For example, the device may be an endoscope containing an endoscopic camera 116 at its tip. In some embodiments, the camera 116 may be connected to an external processing unit using a dedicated shielded cable 1402 of small diameter, where the dedicated shielded cable 1402 runs separately from the sensor array 112 (FPC 112 containing electrical components/sensor 114). In some embodiments, the camera cable 1402 can be wrapped helically inside the device. In the case of a device containing a sensor-array, the camera cable can be wrapped helically inside the device alongside the helically wrapped sensor-array, as schematically shown in FIG. 14.

In some embodiments, instead of using a separate, dedicated camera cable, a camera can be connected through traces on the same FPC 202 as the sensor-array 114, or on a separate dedicated FPC. In this embodiment, the camera power and clock and data signals can be hosted on a same FPC 202 with the sensor-array 114, or on a separate dedicated FPC. In some embodiments, camera clock and digital/analog data signal are shielded to protect them from electrical interference. In some embodiments, dedicated camera power and ground planes are used for shielding of other camera signals (clock and data). In some embodiments, these power and ground planes can be dedicated to the camera, to reduce crosstalk with other signals on the FPC (such as sensor data signals). In some embodiments, the camera and sensors can share the same power and ground planes to reduce FPC size. In some embodiments, the FPC can be a multilayer FPC, for example, 4-layers FPC, such that the addition of camera signals does not necessarily increases it in width. In some embodiments, the final wrapped FPC can consist of two separate sub-FPCs—one for the sensor array and one for the camera. In some embodiments, the two FPCs can be connected using small bridges to create the final wrapped FPC which consists of them both. In some embodiments, connecting the two sub-FPCs using small bridges increases the flexibility of the final integrated wider FPC, compared to a single solid FPC. In some embodiments, the two sub-FPCs can be separate so that the sensor-array and camera traces each lies on a dedicated FPC. In some embodiments, the final FPC may only contain the camera traces (for example, power, ground, clock and data) but may not contain the actual camera component. In this case, the camera may be assembled separately (for example, on another small dedicated FPC) and may be connected to the camera traces on the FPC through short wires between the camera dedicated FPC and exposed camera pads on the sensor-array FPC. In some embodiments, the sensor-array FPC may include both camera traces and camera component pads, and the camera may then be assembled directly on the sensor-array FPC. In some embodiments, as mentioned above the camera may then be spatiality manipulated, such as through folding, and molded into the device's tip as part of an assembly process. In some embodiments, the FPC can be fully automatically assembled using Pick-and-Place machines. This includes all electrical components on the FPC, which may include: a sensor-array (which may consist of a plurality of SMT 3D digital magnetometers), passive components (such as SMT capacitors, resistors, ferrite-beads etc.) and a camera (for example, an SMT camera). Using an automatically or semi-automatically or even manually assembled single integrated FPC for both sensor-array and camera can potentially reduce the device's COGS.

In some embodiments, rather than embedding the FPC inside the wall of an endoscope, the FPC may be wrapped in the wall of a hollow, attachable, shrink-like flexible fixture. In some embodiments, this fixture can then be attached to any existing device, for example, to an endoscopic device, to enhance it with tracking capabilities.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. An elongated device, comprising:
a. a handle;
b. an elongated body comprising a proximal end and a distal end; said proximal end connected to said handle;
c. one or more sensor arrays, each comprising:
i. one or more Flexible Printed Circuits (FPCs); and
ii. a plurality of electronic components positioned on said one or more FPCs;
said one or more sensor arrays being helically wound around said elongated body; and wherein said plurality of electronic components are align in relation to a longitudinal axis of said elongated body.

2. The elongated device according to claim 1, wherein said plurality of electronic components comprise one or more of sensors, capacitors, resistors, integrated circuits, electromagnetic sensors, digital sensors, digital magnetometers and optical sensors.

3. The elongated device according to claim 1, wherein each of said plurality of electronic components is oriented on said one or more FPCs at an orientation angle in relation with a longitudinal axis of said one or more FPCs.

4. The elongated device according to claim 3, wherein said orientation angle is configured so when said one or more FPCs are helically wound around said elongated body, said each of said plurality of electronic components is aligned in relation to said longitudinal axis of said elongated body.

5. The elongated device according to claim 3, wherein said orientation angle is opposite to a winding angle of said one or more sensor arrays.

6. The elongated device according to claim 1, wherein helically winding of said one or more sensor arrays on said elongated body is characterized by a winding angle;
wherein said winding angle is characterized by one or more of:
a. an angle between a longitudinal axis of said one or more FPCs and said longitudinal axis of said elongated body;
b. an angle of 45 degrees;
c. an angle of from 30 degrees to 60 degrees;
d. being fixed along the whole length of said elongated body;
e. changing along the length of said elongated body.

7-9. (canceled)

10. The elongated device according to claim 6, wherein said one or more FPCs comprise a proximal end and a distal end; and wherein said proximal end and/or said distal end are characterized by being cut diagonally at angle that is a same angle as said winding angle.

11. The elongated device according to claim 6, wherein said elongated body is characterized by a stiffness, and wherein said winding angle affects said stiffness;
wherein said stiffness increases when said winding angle is at least one of: closer to 90 degrees or closer to 0 degrees.

12-15. (canceled)

16. The elongated device according to claim 1, wherein all of said plurality of electronic components are positioned on one side of said one or more FPCs.

17. The elongated device according to claim 1, wherein some electronic components of said plurality of electronic components are positioned on one side of said one or more FPCs, while other electronic components of said plurality of electronic components are positioned on another side of said one or more FPCs.

18. The elongated device according to claim 1, wherein said one or more sensor arrays are helically wound around said elongated body having said at least part of said plurality of electronic components facing said elongated body;
wherein said elongated body comprises one or more openings; and wherein said at least part of said plurality of electronic components facing said elongated body are inserted within said one or more openings; and
wherein said at least part of said plurality of electronic components are shielded from external electrical interference when inserted in said one or more openings.

19-20. (canceled)

21. The elongated device according to claim 1, wherein said one or more FPCs are characterized by one or more of:
a. being adhered to said elongated body by at least one adhesive;
b. being widen around a location where each of said plurality of electronic components are positioned on said one or more FPCs;
c. being narrow between locations where each of said plurality of electronic components are positioned on said one or more FPCs;
d. being multilayer FPCs;
e. only contain camera traces but do not contain an actual camera component;
f. being connected to a component in said handle.

22-23. (canceled)

24. The elongated device according to claim 1, further comprising one or more conductive wirings helically wound around said elongated body; and wherein at least one of the following is true:
a. a location of said one or more conductive wirings helically wound around said elongated body is different as a location where said one or more sensor arrays are helically wound around said elongated body;
b. said one or more conductive wirings are one or more insulated conductive wirings;
c. one or more electronic components are connected to said one or more conductive wiring;
d. said one or more conductive wirings are printed conductive ink.

25-28. (canceled)

29. The elongated device according to claim 1, wherein electronic components from said plurality of electronic components are positioned on different FPCs from said one or more FPCs.

30. The elongated device according to claim 2, further comprising a dedicated FPC for said optical sensors.

31. The elongated device according to claim 1, wherein all of said plurality of electronic components are positioned on a same FPC.

32. The elongated device according to claim 1, wherein said one or more FPCs comprise one or more of:
a. small copper weights;
b. small holes to allow plastic materials to flow through said one or more FPCs in a reflow process;
c. solder mask between solder pads;
d. a male connector at least one of said proximal end and a distal end of said one or more FPCs;
e. one or more creases in one or multiple connecting areas between soldered components.

33. (canceled)

34. The elongated device according to claim 1, wherein at least some of said plurality of electronic components are SMT components; and wherein said SMT components are soldered to plated holes in said one or more FPCs.

35. (canceled)

36. The elongated device according to claim 1, further comprising at least one camera; and wherein at least one of the following is true:
a. camera's power and clock and data signals are hosted on a same FPC with said one or more sensor arrays;
b. camera's power and clock and data signals are hosted on a separate dedicated FPC:
c. camera clock and digital/analog data signal are shielded from electrical interference;
d. further comprising dedicated camera power and ground planes configured for shielding said elongated device from other camera signals.

37-42. (canceled)

43. The elongated device according to claim 10, further comprising an electrical connector attached to at least one of said proximal end and a distal end of said one or more FPCs.

44. (canceled)

45. The elongated device according to claim 1, further comprising an Inter-Integrated Circuit (I2C) or Improved Inter-Integrated Circuit (I3C) bus.

46-50. (canceled)
